(19) United States
(12) Patent Application Publication
(10) Pub. No.: US 2025/0090530 A1
(43) Pub. Date: Mar. 20, 2025

(54) COMBINATIONS FOR THE TREATMENT OF CANCER

(71) Applicant: Terns Pharmaceuticals, Inc., Foster City, CA (US)

(72) Inventors: Benjamin M. PARSONS, Foster City, CA (US); Christopher T. JONES, Foster City, CA (US); Jeffrey R. JASPER, Mountain View, CA (US)

(21) Appl. No.: 18/820,700

(22) Filed: Aug. 30, 2024

Related U.S. Application Data
(60) Provisional application No. 63/535,881, filed on Aug. 31, 2023.

(51) Int. Cl. A61K 31/506; A61K 31/5025; A61P 35/02; C12Q 1/68

(52) U.S. Cl. CPC A61K 31/506; A61K 31/5025; A61P 35/02; C12Q 1/68

(57) ABSTRACT

Provided herein are combinations for inhibiting tyrosine kinase activity of a protein selected from Abelson protein (ABL1), Abelson-related protein (ABL2), or a chimeric protein BCR-ABL1, compositions thereof, methods of inhibiting tyrosine kinase enzymatic activity of a protein selected from Abelson protein (ABL1), Abelson-related protein (ABL2), or a chimeric protein BCR-ABL1, and methods for treating diseases wherein modulation of BCR-ABL1 activity prevents, inhibits, or ameliorates the pathology and/or symptomology of the disease.

### CROSS REFERENCE TO RELATED APPLICATIONS

The present application claims priority to and the benefit of U.S. Provisional Patent Application No. 63/535,881, filed on Aug. 31, 2023, which is incorporated herein by reference in its entirety for all purposes.

### REFERENCE TO AN ELECTRONIC SEQUENCE LISTING

The contents of the electronic sequence listing (TRPH_048_001WO_SeqList_ST26.xml; Size: 44,747 bytes; and Date of Creation: Aug. 29, 2024) are herein incorporated by reference in its entirety.

### BACKGROUND

In chronic myeloid leukemia (CML) the Philadelphia chromosome (Ph) is formed through a reciprocal translocation between chromosomes 9 and 22 in myeloid progenitor cells. This chromosome carries the BCR-ABL1 oncogene which encodes the chimeric BCR-ABL1 protein. Drugs that inhibit the tyrosine kinase activity of BCR-ABL1 via an ATP competitive mechanism, such as Gleevec®/Glivec® (imatinib), Tasigna® (nilotinib), Iclusig® (ponatinib) and Sprycel® (dasatinib), may be effective in treating CML; however, some patients relapse due to the emergence of drug-resistant clones or drug intolerance. Thus, there is a need to provide additional therapeutic options.

### SUMMARY

The present disclosure relates to a method of treating cancer in a subject in need thereof, comprising administering a BCR:ABL1 inhibitor in combination with a tyrosine-kinase inhibitor, wherein the BCR:ABL1 inhibitor is a compound of formula (I):

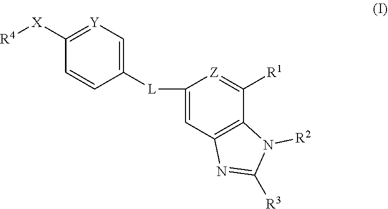

or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷;
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-.

In some embodiments, the BCR:ABL1 inhibitor is a compound is of formula (IA-1):

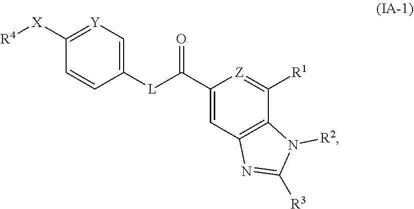

or a pharmaceutically acceptable salt thereof.

The present disclosure relates to using (R)—N-(4-(chlorodifluoromethoxy)phenyl)-2-(difluoromethyl)-1-(1-hydroxypropan-2-yl)-7-(pyrimidin-5-yl)-1H-benzo[d]imidazole-5-carboxamide (Compound A):

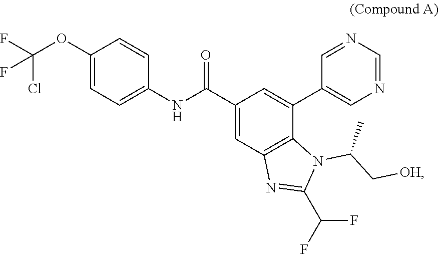 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof, in combination with a tyrosine-kinase inhibitor, for the treatment of cancer in a subject in need thereof.

The present disclosure relates to using Compound A:

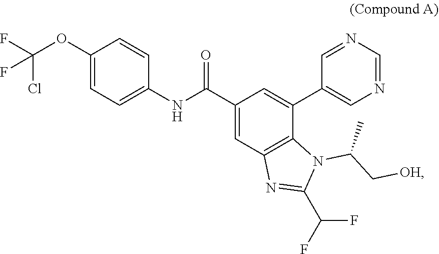 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof, in combination with a tyrosine-kinase inhibitor selected from imatinib, nilotinib, dasatinib, bosutinib, ponatinib and bafetinib, for the treatment of cancer in a subject in need thereof.

The present disclosure also relates to a method of treating cancer in a subject in need thereof, comprising administering Compound A, or a pharmaceutically acceptable salt thereof, in combination with a tyrosine-kinase inhibitor.

The present disclosure also relates to a method of treating cancer in a subject in need thereof, comprising administering Compound A, or a pharmaceutically acceptable salt thereof, in combination with a tyrosine-kinase inhibitor selected from imatinib, nilotinib, dasatinib, bosutinib, ponatinib and bafetinib.

The present disclosure further relates to use of Compound A, or a pharmaceutically acceptable salt thereof, in combination with a tyrosine-kinase inhibitor, for the preparation of a medicament for the treatment of cancer in a subject in need thereof.

The present disclosure further relates to use of Compound A, or a pharmaceutically acceptable salt thereof, in combination with a tyrosine-kinase inhibitor selected from imatinib, nilotinib, dasatinib, bosutinib, ponatinib and bafetinib, for the preparation of a medicament for the treatment of cancer in a subject in need thereof.

The present disclosure relates to using Compound A or a pharmaceutically acceptable salt thereof, in combination with a tyrosine-kinase inhibitor, for the treatment of cancer in a subject in need thereof.

The present disclosure relates to using Compound A or a pharmaceutically acceptable salt thereof, in combination with a tyrosine-kinase inhibitor selected from asciminib, imatinib, nilotinib, dasatinib, bosutinib, ponatinib and bafetinib, for the treatment of cancer in a subject in need thereof.

The present disclosure also relates to a method of treating cancer, comprising administering Compound A, or a pharmaceutically acceptable salt thereof, in combination with a tyrosine-kinase inhibitor.

The present disclosure also relates to a method of treating cancer, comprising administering Compound A, or a pharmaceutically acceptable salt thereof, in combination with a tyrosine-kinase inhibitor, wherein the tyrosine kinase inhibitor binds to the active site on the tyrosine-kinase.

The present disclosure also relates to a method of treating cancer, comprising administering Compound A, or a pharmaceutically acceptable salt thereof, in combination with a tyrosine-kinase inhibitor selected from asciminib, imatinib, nilotinib, dasatinib, bosutinib, ponatinib and bafetinib.

The present disclosure relates to using Compound A or a pharmaceutically acceptable salt thereof, in combination with a tyrosine-kinase inhibitor, for the treatment of lymphoma or leukemia in a subject in need thereof.

The present disclosure relates to using Compound A or a pharmaceutically acceptable salt thereof, in combination with a tyrosine-kinase inhibitor selected from asciminib, imatinib, nilotinib, dasatinib, bosutinib, ponatinib and bafetinib, for the treatment of lymphoma or leukemia in a subject in need thereof.

The present disclosure also relates to a method of treating lymphoma or leukemia in a subject in need thereof, comprising administering Compound A, or a pharmaceutically acceptable salt thereof, in combination with a tyrosine-kinase inhibitor.

The present disclosure also relates to a method of treating lymphoma or leukemia in a subject in need thereof, comprising administering Compound A, or a pharmaceutically acceptable salt thereof, in combination with a tyrosine-kinase inhibitor selected from asciminib, imatinib, nilotinib, dasatinib, bosutinib, ponatinib and bafetinib.

The present disclosure further relates to use of Compound A, or a pharmaceutically acceptable salt thereof, in combination with a tyrosine-kinase inhibitor, for the preparation of a medicament for the treatment of lymphoma or leukemia in a subject in need thereof.

The present disclosure further relates to use of Compound A, or a pharmaceutically acceptable salt thereof, in combination with a tyrosine-kinase inhibitor selected from asciminib, imatinib, nilotinib, dasatinib, bosutinib, ponatinib and bafetinib, for the preparation of a medicament for the treatment of lymphoma or leukemia in a subject in need thereof.

The present disclosure further relates to a method of inhibiting tyrosine kinase enzymatic activity of a protein selected from the group consisting of Abelson protein (ABL1), Abelson-related protein (ABL2), and a chimeric protein BCR-ABL1, comprising contacting an effective amount of Compound A in combination with an effective amount of atyrosine-kinase inhibitor, with the protein.

The present disclosure further relates to a method of treating a disease, wherein modulation of BCR-ABL1 activity prevents, inhibits, or ameliorates the pathology and/or symptomology of the disease, in a patient, comprising administering to the patient a therapeutically effective amount of Compound A in combination with a therapeutically effective amount of a tyrosine-kinase inhibitor.

The present disclosure further relates to a method of treating leukemia in a patient comprising administering to the patient a therapeutically effective amount of Compound A in combination with a therapeutically effective amount of a tyrosine-kinase inhibitor, wherein the leukemia is chronic myeloid leukemia (CML), acute myeloid leukemia (AML), or acute lymphoblastic leukemia (ALL).

In some embodiments, the combination comprises administering Compound A and a tyrosine-kinase inhibitor selected from asciminib, imatinib, nilotinib, dasatinib, bosutinib, ponatinib and bafetinib at a ratio of about 1:1, about 1:2, about 1:3, about 1:4, about 1:5, about 2:1, about 2:3, about 2:5, about 3:1, about 3:2, about 3:4, about 3:5, about 4:1, about 4:3, or about 4:5.

In some embodiments, the combination comprises a BCR:ABL1 inhibitor and a tyrosine-kinase inhibitor, wherein the BCR:ABL1 inhibitor is a compound of formula (I):

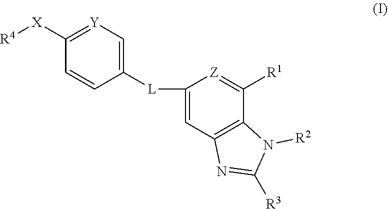

or a tautomer or an N-oxide thereof, or a pharmaceutically acceptable salt of any of the foregoing; and at least one pharmaceutically acceptable excipient.

In some embodiments, the combination is used for treating leukemia in a patient comprising administering to the patient a therapeutically effective amount of the BCR:ABL1 inhibitor and a therapeutically effective amount of the tyrosine-kinase inhibitor, wherein the leukemia is chronic myeloid leukemia (CML), acute myeloid leukemia (AML), or acute lymphoblastic leukemia (ALL).

In some embodiments, the leukemia is CML or ALL, and the tyrosine-kinase inhibitor is selected from the group selected from asciminib, imatinib, nilotinib, dasatinib, bosutinib, ponatinib and bafetinib.

In some embodiments, the CML is resistant to standard-of-care treatment.

In some embodiments, the CML is resistant to treatment with one or more of imatinib, ponatinib, nilotinib, and dasatinib.

In some embodiments, the AML is secondary AML, which develops after myelodysplastic syndromes (MDS) or myeloproliferative neo-plasms (MPN).

### BRIEF DESCRIPTION OF THE FIGURES

FIG. 1 is a schematic representation of allosteric inhibition of the BCR::ABL1 oncoprotein.

FIG. 2 depicts the potency and selectivity of Compound A and asciminib against BCR::ABL1+ and BCR::ABL1-cell lines.

FIG. 3 depicts representative cytotoxicity curves (L) for molar combinations of Compound A and dasatinib (top) and ponatinib (bottom). Right panels are representative synergy isobolograms set against the IC₇₅ values for single agents and fixed molar combinations with dasatinib (top) or ponatinib (bottom). Points falling below the blue additivity line indicate synergistic interaction.

FIG. 4A depicts a representative heat map of K562 cells treated with Compound A and dasatinib. Higher values indicate stronger synergy.

FIG. 4B depicts a representative heat map of BaF3-BCR-ABL-T315I cells treated with Compound A and ponatinib. Higher values indicate stronger synergy.

FIG. 5A depicts a representative heat map of BaF3-BCR-ABL-T315I cells treated with Compound A and ponatinib. Higher values indicate stronger synergy.

FIG. 5B depicts a representative heat map of BaF3-BCR-ABL-T315I cells treated with Compound A and dasatinib. Higher values indicate stronger synergy.

FIG. 6A depicts representative cytotoxicity curves (L) for molar combinations of Compound A and dasatinib. The right panel is representative synergy isobolograms set against the IC₇₅ values for single agents and fixed molar combinations with dasatinib. Points falling below the blue additivity line indicate synergistic interaction.

FIG. 6B depicts representative cytotoxicity curves (L) for molar combinations of Compound A and dasatinib. The right panel is representative synergy isobolograms set against the IC₇₅ values for single agents and fixed molar combinations with dasatinib. Points falling below the blue additivity line indicate synergistic interaction.

FIG. 7 depicts tumor measured on day 15 of an in vivo BCR::ABL1-T315I CML model in which animals were treated with a combination of Compound A and second TKIs.

FIG. 8 depicts the change in median tumor volume over 15 days of an in vivo BCR::ABL1-T315I CML model in which animals were treated with a combination of Compound A and second TKIs.

### DETAILED DESCRIPTION

### Definitions

As used herein, the following definitions shall apply unless otherwise indicated. Further, if any term or symbol used herein is not defined as set forth below, it shall have its ordinary meaning in the art.

As used herein, in the context of a tyrosine kinase protein, “active site” is intended to refer to the region of the tyrosine kinase protein where a phosphate group is catalytically transferred from adenosine triphosphate (ATP) to substrate.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Effective amount” or dose of a compound or a composition, refers to that amount of the compound or the composition that results in an intended result as desired based on the disclosure herein. Effective amounts can be determined by standard pharmaceutical procedures in cell cultures or experimental animals, e.g., and without limitation, by determining the LD₅₀ (the dose lethal to 50% of the population) and the ED₅₀ (the dose therapeutically effective in 50% of the population).

“Combination therapy” or “combination treatment” refers to the use of two or more drugs or agents in treatment, e.g., the use of a compound of formula (I), (I-i), (IA), (IA-1), (IIA), (IIB), (IIE), or (Id), such as Compound A, as utilized herein together with a tyrosine-kinase inhibitor useful to treat cancer, such as lymphoma and leukemia, and symptoms and manifestations of each thereof is a combination therapy. Administration in “combination” refers to the administration of two agents (e.g., a compound of formula (I), (I-i), (IA), (IA-1), (IIA), (IIB), (IIE), or (Id), such as Compound A, as utilized herein, and a tyrosine-kinase inhibitor) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that a compound of formula (I), (I-i), (IA), (IA-1), (IIA), (IIB), (IIE), or (Id), such as Compound A, can be administered in combination therapy with a tyrosine-kinase inhibitor in accordance with the present invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “subject” ot “patient” includes human and non-human animals, as well as cell lines, cell cultures, tissues, and organs. In some embodiments, the subject is a mammal. The mammal can be e.g., a human or appropriate non-human mammal, such as primate, mouse, rat, hamster, dog, rabbit, cat, cow, horse, goat, camel, sheep, guinea pig or a pig. The subject can also be a bird or fowl. In some embodiments, the subject is a human. In some embodiments, patient is a human.

The term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

The term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

The term “co-administration” or the like, as used herein, is meant to encompass administration of the selected therapeutic agents to a single patient and are intended to include treatment regimens in which the agents are administered by the same or different route of administration or at the same or different time.

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than employing the corresponding drug. For illustration and without limitation, prodrugs include, carboxy esters, linear and cyclic phosphate esters and phosphoramide and phosphoramidates, carbamates, preferably phenolic carbamates (i.e., carbamates where the hydroxy group is part of an aryl or heteroaryl moiety, where the aryl and heteroaryl may be optionally substituted), and the likes.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. “Therapeutically effective amount” or dose of a compound or a composition refers to that amount of the compound or the composition that results in reduction or inhibition of symptoms or a prolongation of survival in a patient. The results may require multiple doses of the compound or the composition.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

An “isotopomer” of a compound is a compound in which one or more atoms of the compound have been replaced with isotopes of those same atoms. For example, where H has been replaced by D or T, or ¹²C has been replaced by ¹¹C or ¹⁴N has been replaced by ¹⁵N. For example, and without limitation, replacement of with D can in some instances lead to reduced rates of metabolism and therefore longer half-lives. Replacement of H with T can provide radioligands potentially useful in binding studies. Replacement of ¹²C with the short-lived isotope ¹¹C can provide ligands useful in Positron Emission Tomography (PET) scanning. Replacement of ¹⁴N with ¹⁵N provides compounds that can be detected/monitored by ¹⁵N NMR spectroscopy. For example, an isotopomer of a compound containing —CH₂CH₃ is that compound but containing —CD₂CD₃ instead of the —CH₂CH₃.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring-NH-moiety and a ring ═N— moiety such as pyrazoles, imidazoles, benzimidazoles, triazoles, and tetrazoles.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), 1-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester) amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester) amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester) amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxyl or thiol substitution is not attached to an acetylenic carbon atom.

“Alkoxy” refers to the group-O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group-O-(substituted alkyl) wherein substituted alkyl is defined herein. Preferred substituted alkyl groups in-O-(substituted alkyl) include halogenated alkyl groups and particularly halogenated methyl groups such as trifluoromethyl, difluromethyl, fluoromethyl and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NR³⁰C(O)alkyl, —NR³⁰C(O)substituted alkyl, —NR³⁰C(O)cycloalkyl, —NR³⁰C(O)substituted cycloalkyl, —NR³⁰C(O)alkenyl, —NR³⁰C(O)substituted alkenyl, alkoxy, substituted alkynyl-NR²⁰C(O)alkynyl, —NR³⁰C(O)substituted alkynyl, —NR³⁰C(O)aryl, —NR³⁰C(O)substituted aryl, —NR³⁰C(O)heteroaryl, —NR³⁰C(O)substituted heteroaryl, —NR³⁰C(O)heterocyclic, and —NR³⁰C(O)substituted heterocyclic wherein R³⁰ is hydrogen, alkyl, substituted alkyl, aryl, substituted aryl, heteroaryl, substituted heteroaryl, cycloalkyl, or substituted cycloalkyl; and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, substituted heterocyclic, arylamino, substituted arylamino, heteroarylamino, substituted heteroarylamino, cycloalkylamino, substituted cycloalkylamino, heterocycloalkylamino, substituted heterocyclylamino, sulfonylamino, and substituted sulfonyl and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonyl” refers to the group —C(S)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR³⁰C(O)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the group —NR³⁰C(S)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the group —O—C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the group —SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the group —O—SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the group —NR³⁰—SO₂NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Amidino” refers to the group —C(═NR³⁵)NR³³R³⁴ where R³³, R³⁴, and R³⁵ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3 (4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino carboxyl, carboxyl ester, (carboxyl ester) amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Arylamino” refers to the group —NR³⁷(aryl), where aryl is as defined herein and R³⁷ is hydrogen, alkyl, or substituted alkyl.

“Substituted arylamino” refers to the group —NR³⁷ (substituted aryl), where R³⁷ is hydrogen, alkyl, or substituted alkyl where substituted aryl is as defined herein.

“Carbonyl” refers to the divalent group —C(O)— which is equivalent to —C(═O)—.

“Carboxy” or “carboxyl” refers to-COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O— substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester) amino” refers to the group —NR³⁰—C(O)O-alkyl, —NR³⁰—C(O)O-substituted alkyl, —NR³⁰—C(O)O-alkenyl, —NR³⁰—C(O)O-substituted alkenyl, —NR³⁰—C(O)O-alkynyl, —NR³⁰—C(O)O-substituted alkynyl, —NR³⁰—C(O)O-aryl, —NR³⁰—C(O)O-substituted aryl, —NR³⁰—C(O)O-cycloalkyl, —NR³⁰—C(O)O-substituted cycloalkyl, —NR³⁰—C(O)O-heteroaryl, —NR³⁰—C(O)O-substituted heteroaryl, —NR³⁰—C(O)O-heterocyclic, and —NR³⁰—C(O)O-substituted heterocyclic wherein R³⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylamino” refers to the group —NR³⁷ (cycloalkyl) where R³⁷ is hydrogen, alkyl, or substituted alkyl.

“Substituted cycloalkylamino” refers to the group —NR³⁷ (substituted cycloalkyl) where R³⁷ is hydrogen, alkyl, or substituted alkyl and substituted cycloalkyl is as defined herein.

“Cycloalkylthio” refers to —S-cycloalkyl.

“Substituted cycloalkylthio” refers to —S-(substituted cycloalkyl).

“Guanidino” refers to the group —NHC(═NH)NH₂.

“Substituted guanidino” refers to —NR³⁶C(═NR³⁶)N(R³⁶)₂ where each R³⁶ is independently selected from the group consisting of hydrogen, alkyl, substituted alkyl, aryl, substituted aryl, heteroaryl, substituted heteroaryl, heterocyclic, and substituted heterocyclic and two R³⁶ groups attached to a common guanidino nitrogen atom are optionally joined together with the nitrogen bound thereto to form a heterocyclic or substituted heterocyclic group, provided that at least one R³⁶ is not hydrogen, and wherein said substituents are as defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heteroarylamino” refers to the group —NR³⁷(heteroaryl) where R³⁷ is hydrogen, alkyl, or substituted alkyl.

“Substituted heteroarylamino” refers to the group —NR³⁷ (substituted heteroaryl), where R³⁷ is hydrogen, alkyl, or substituted alkyl and substituted heteroaryl is defined as herein.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

“Heterocyclylamino” refers to the group —NR³⁷ (heterocyclyl) where R³⁷ is hydrogen, alkyl, or substituted alkyl.

“Substituted heterocyclylamino” refers to the group —NR³⁷ (substituted heterocyclyl), where R³⁷ is hydrogen, alkyl, or substituted alkyl and substituted heterocyclyl is defined as herein.

“Nitro” refers to the group —NO₂.

“Oxo” refers to the atom (═O) or (O).

“Spiro ring systems” refers to bicyclic ring systems that have a single ring carbon atom common to both rings.

“Sulfinyl” refers to the divalent group —S(O)— or —S(═O)—.

“Sulfonyl” refers to the divalent group —S(O)₂— or —S(═O)₂—.

“Substituted sulfonyl” refers to the group —SO₂-alkyl, —SO₂-substituted alkyl, —SO₂—OH, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—. Preferred substituted alkyl groups on the substituted alkyl-SO₂— include halogenated alkyl groups and particularly halogenated methyl groups such as trifluoromethyl, difluromethyl, fluoromethyl and the like.

“Sulfonyloxy” or “substituted sulfonyloxy” refers to the group —OSO₂-alkyl, —OSO₂-substituted alkyl, —OSO₂—OH, —OSO₂-alkenyl, —OSO₂-substituted alkenyl, —OSO₂-cycloalkyl, —OSO₂-substituted cycloalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Sulfonylamino” refers to the group —NR³⁷ (substituted sulfonyl) where R³⁷ is hydrogen, alkyl, or substituted alkyl and substituted sulfonyl is as defined here.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Mercapto” or “thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein. Preferred substituted alkyl groups on —S-(substituted alkyl) include halogenated alkyl groups and particularly halogenated methyl groups such as trifluoromethyl, difluromethyl, fluoromethyl and the like.

“Vinyl” refers to unsaturated hydrocarbon radical —CH═CH₂, derived from ethylene.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

The term “optionally substituted” refers to a substituted or unsubstituted group. The substituted group may be substituted with one or more substituents, such as e.g., 1, 2, 3, 4 or 5 substituents. Preferably, the substituents are selected from the functional groups provided herein. In certain more preferred embodiments, the substituents are selected from oxo, halo, —CN, NO₂, —CO₂R¹⁰⁰, —SR¹⁰⁰, —OR¹⁰⁰, —SOR¹⁰⁰, —SO₂R¹⁰⁰, —NR¹⁰¹R¹⁰², —CONR¹⁰¹R¹⁰², —SO₂NR¹⁰¹R¹⁰², C₁-C₆ alkyl, C₁-C₆ alkoxy, —CR¹⁰⁰—C(R¹⁰⁰)₂, —CCR¹⁰⁰, C₃-C₁₀ cycloalkyl, C₄-C₁₀ heterocyclyl, C₆-C₁₄ aryl and C₅-C₁₂ heteroaryl, wherein each R¹⁰⁰ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. More preferably, the substituents are selected from the group consisting of chloro, fluoro, —OCH₃, methyl, ethyl, iso-propyl, cyclopropyl, —OCF₃, —CF₃ and —OCHF₂.

R¹⁰¹ and R¹⁰² independently are hydrogen; C₁-C₈ alkyl, optionally substituted with —CO₂H or an ester thereof, C₁-C₆ alkoxy, oxo, —CR¹⁰³═C(R¹⁰³)₂, —CCR, C₃-C₁₀ cycloalkyl, C₃-C₁₀ heterocyclyl, C₆-C₁₄ aryl, or C₂-C₁₂ heteroaryl, wherein each R¹⁰³ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each cycloalkyl, heterocyclyl, aryl, or heteroaryl is optionally substituted with 1-3 alkyl groups or 1-3 halo groups, or R¹⁰¹ and R¹⁰² together with the nitrogen atom they are attached to form a 5-7 membered heterocycle.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to-substituted aryl-(substituted aryl)-substituted aryl.

In some embodiments of a substituted moiety, the moiety is substituted with a group that may also be substituted with a further group, but the further group cannot be additionally substituted. For example, in some embodiments of “substituted alkyl”, the alkyl moiety is substituted with a group that may be further substituted (e.g., substituted alkoxy, substituted amino, substituted aryl, substituted aryloxy, substituted arylthio, substituted arylamino, substituted heteroarylamino, substituted cycloalkylamino, substituted heterocyclylamino, substituted cycloalkyl, substituted cycloalkyloxy, substituted cycloalkylthio, substituted guanidino, substituted heteroaryl, substituted heteroaryloxy, substituted heteroarylthio, substituted heterocyclic, substituted heterocyclyloxy, substituted heterocyclylthio, substituted sulfonyl, substituted alkylthio), but the substituted alkoxy, substituted amino, substituted aryl, substituted aryloxy, substituted arylthio, substituted arylamino, substituted heteroarylamino, substituted cycloalkylamino, substituted heterocyclylamino, substituted cycloalkyl, substituted cycloalkyloxy, substituted cycloalkylthio, substituted guanidino, substituted heteroaryl, substituted heteroaryloxy, substituted heteroarylthio, substituted heterocyclic, substituted heterocyclyloxy, substituted heterocyclylthio, substituted sulfonyl or substituted alkylthio on the alkyl moiety is not substituted with a moiety that is itself further substituted. Although “substituted alkyl” is provided as an example, such an embodiment is intended for each substituted moiety described herein.

In some embodiments of a substituted moiety, the moiety is substituted with a group that is not further substituted. Thus, in some embodiments, “substituted alkyl” is an alkyl moiety substituted with one or more, and in some aspects, 1 or 2 or 3 or 4 or 5 moieties independently selected from the group consisting of alkoxy, acyl, acylamino, acyloxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, arylamino, heteroarylamino, cycloalkylamino, heterocycloalkylamino, carboxyl, carboxyl ester, (carboxyl ester) amino, (carboxyl ester)oxy, cyano, cycloalkyl, cycloalkyloxy, cycloalkylthio, guanidino, halo, hydroxy, heteroaryl, heteroaryloxy, heteroarylthio, heterocyclic, heterocyclyloxy, heterocyclylthio, nitro, SO₃H, sulfonyloxy, sulfonylamino, thioacyl, thiol, and alkylthio. Although “substituted alkyl” is provided as an example, such an embodiment is intended for each substituted moiety described herein.

It is understood that the above definitions are not intended to include impermissible substitution patterns (e.g., methyl substituted with 4 fluoro groups). Such impermissible substitution patterns are well known to the skilled artisan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### BCR:ABL1 Inhibitors

The present disclosure relates to using a BCR:ABL1 inhibitor in combination with a tyrosine-kinase inhibitor, for the treatment of cancer in a subject in need thereof.

In some embodiments, the BCR:ABL1 inhibitor is a compound of formula (I):

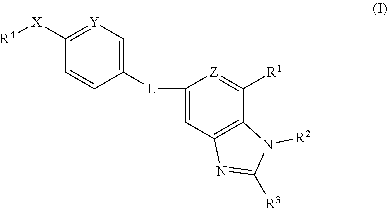

or a tautomer or an N-oxide thereof, or an isotopomer of each thereof, or a prodrug of each of the above, or a stereoisomer of the aforesaid, or a pharmaceutically acceptable salt of each of the foregoing, or a solvate of each of the preceding, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, or NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷, or
R² and R³ together with the intervening atoms form cycloalkyl or heterocycloalkyl, preferably an optionally substituted C₃-C₈ cycloalkyl or an optionally substituted 4-10 membered heterocycloalkyl;
R⁴ is optionally substituted C₁-C₆ alkyl, preferably C₁-C₃ haloalkyl, such as CF₃ or CF₂Cl, optionally substituted C₂-C₆ alkenyl, optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; or
R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-.

In some aspects, the BCR:ABL1 inhibitor is a compound of formula (I-i):

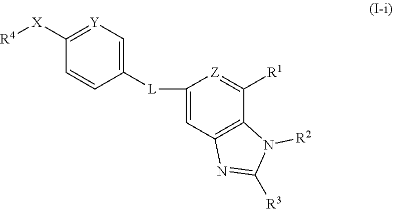

or a tautomer or an N-oxide thereof, or an isotopomer of each thereof, or a prodrug of each of the above, or a stereoisomer of the aforesaid, or a pharmaceutically acceptable salt of each of the foregoing, or a solvate of each of the preceding, wherein:
L is —NH—CO—, —CO—NH—, or —NH—SO₂—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, or NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷, or
R² and R³ together with the intervening atoms form cycloalkyl or heterocycloalkyl, preferably an optionally substituted C₃-C₈ cycloalkyl or an optionally substituted 4-10 membered heterocycloalkyl;
R⁴ is optionally substituted C₁-C₆ alkyl, preferably C₁-C₃ haloalkyl, such as CF₃ or CF₂Cl, optionally substituted C₂-C₆ alkenyl, optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; or
R⁶ and R⁷ together with the nitrogen to which they are attached form an optionally substituted 4-7 membered heterocycle.

In some embodiments, the BCR:ABL1 inhibitor is a compound is of formula (IA):

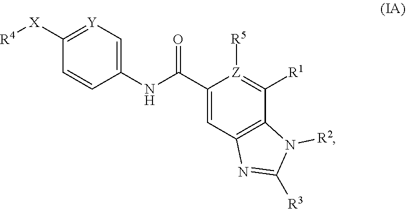

or a pharmaceutically acceptable salt thereof.

It is understood that when Z is N, then R⁵ is absent. Similarly, it is understood that when R⁵ is present, then R⁵ is bound to a carbon atom in the aryl ring such that Z is CR⁵.

In some embodiments, the BCR:ABL1 inhibitor is a compound is of formula (IA-1):

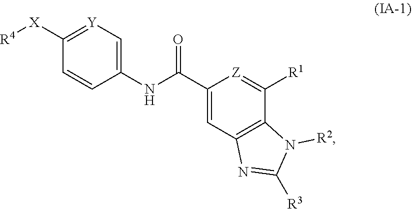

or a pharmaceutically acceptable salt thereof.

In some embodiments, the BCR:ABL1 inhibitor is a compound is selected from formula (IIA), (IIB), or (IIE):

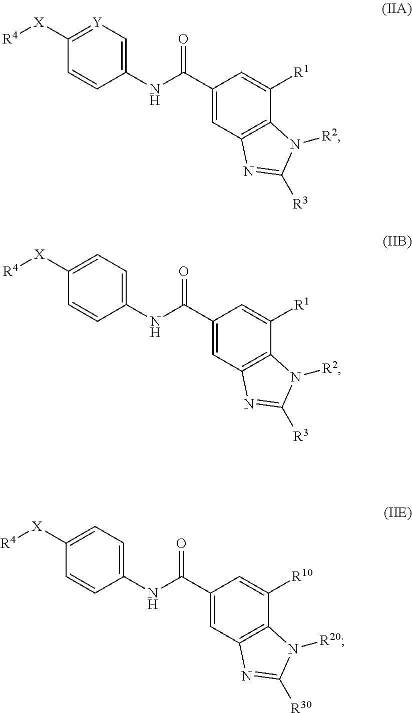

or a pharmaceutically acceptable salt thereof
wherein R¹⁰ is an optionally substituted 5-6 membered heteroaryl, preferably the heteroaryl moiety has up to 2 ring nitrogen atoms;
R²⁰ is optionally substituted C₁-C₃ alkyl, optionally substituted C₃-C₄ cycloalkyl, or optionally substituted 4-6 membered heterocycloalkyl, preferably, R²⁰ is methyl, optionally substituted isopropyl, or cyclopropyl;
R³⁰ is H, optionally substituted C₁-C₃ alkyl, optionally substituted C₃-C₄ cycloalkyl, or optionally substituted 5-6 membered heterocycloalkyl, preferably optionally substituted cyclopropyl; and
the remaining variables are defined as herein.

In some embodiments, the BCR:ABL1 inhibitor is a compound of formula (Id):

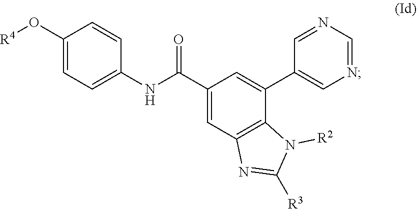

or a pharmaceutically acceptable salt thereof, wherein the remaining variables are defined as herein.

In some embodiments, the BCR:ABL1 inhibitor is Compound A:

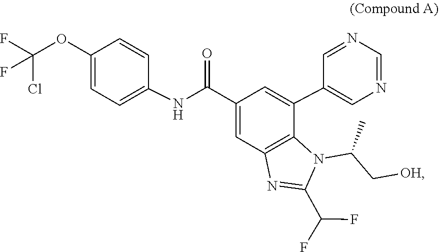 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof.

Suitable BCR:ABL1 inhibitors that can be used in accordance with the methods described herein include, but are not limited to a compound of formula (I), (I-i), (IA), (IA-1), (IIA), (IIB), (IIE), or (Id), such as Compound A, or a pharmaceutically acceptable salt.

The compounds of of formula (I), (I-i), (IA), (IA-1), (IIA), (IIB), (IIE), or (Id), including Compound A, are disclosed in U.S. Pat. No. 10,889,571, the content of which is incorporated by reference in its entirety, and specifically with respect to the compound of formula (I), (I-i), (IA), (IA-1), (IIA), (IIB), (IIE), or (Id), including Compound A, or a pharmaceutically acceptable salt thereof, as well as methods of making and using same.

This disclosure also includes all salts, such as pharmaceutically acceptable salts, of compounds referred to herein. This disclosure also includes any or all of the stereochemical forms, including any enantiomeric or diastereomeric forms, and any tautomers or other forms, such as N-oxides, solvates, prodrugs, or isotopomers, of the compounds described. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

### Tyrosine-Kinase Inhibitor

In some embodiments, the tyrosine-kinase inhibitor is selected from imatinib, nilotinib, dasatinib, bosutinib, ponatinib and bafetinib.

In some embodiments, the tyrosine-kinase inhibitor binds to the active site of the tyrosine-kinase.

In some embodiments, the tyrosine-kinase inhibitor is imatinib.

In some embodiments, the tyrosine-kinase inhibitor is nilotinib.

In some embodiments, the tyrosine-kinase inhibitor is dasatinib.

In some embodiments, the tyrosine-kinase inhibitor is bosutinib.

In some embodiments, the tyrosine-kinase inhibitor is ponatinib.

In some embodiments, the tyrosine-kinase inhibitor is bafetinib.

Combination of a BCR:ABL1 Inhibitor with a Tyrosine Kinase Inhibitor

Disclosed herein, in certain embodiments, are combinations comprising a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib).

Further disclosed herein, in certain embodiments, are pharmaceutical combinations comprising a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib).

Disclosed herein, in certain embodiments, are combinations comprising Compound A and a tyrosine-kinase inhibitor selected from asciminib, imatinib, nilotinib, dasatinib, bosutinib, ponatinib and bafetinib.

Further disclosed herein, in certain embodiments, are pharmaceutical combinations comprising Compound A and a tyrosine-kinase inhibitor selected from asciminib, imatinib, nilotinib, dasatinib, bosutinib, ponatinib and bafetinib.

In some embodiments, Compound A and the tyrosine-kinase inhibitor selected from asciminib, imatinib, nilotinib, dasatinib, bosutinib, ponatinib and bafetinib are co-administered concurrently (e.g., simultaneously, essentially simultaneously or within the same treatment protocol) or sequentially.

In some embodiments, the co-administration of Compound A and the tyrosine-kinase inhibitor selected from asciminib, imatinib, nilotinib, dasatinib, bosutinib, ponatinib and bafetinib increases the bioavailability of Compound A. In some embodiments, the co-administration of Compound A and the tyrosine-kinase inhibitor selected from asciminib, imatinib, nilotinib, dasatinib, bosutinib, ponatinib and bafetinib increases the Cₘₐₓ of Compound A. In some embodiments, the co-administration of Compound A and the tyrosine-kinase inhibitor selected from asciminib, imatinib, nilotinib, dasatinib, bosutinib, ponatinib and bafetinib increases the AUC of Compound A. In some embodiments, the co-administration of Compound A and the tyrosine-kinase inhibitor selected from asciminib, imatinib, nilotinib, dasatinib, bosutinib, ponatinib and bafetinib increases the T_{1/2} of Compound A.

Compositions or therapies disclosed herein may be administered individually to a patient or may be administered in combination (e.g. simultaneously, sequentially or separately). In some embodiments, Compound A is administered in advance of the tyrosine-kinase inhibitor selected from asciminib, imatinib, nilotinib, dasatinib, bosutinib, ponatinib and bafetinib In some embodiments, the tyrosine-kinase inhibitor selected from asciminib, imatinib, nilotinib, dasatinib, bosutinib, ponatinib and bafetinib is administered before Compound A.

In some embodiments, Compound A and the tyrosine-kinase inhibitor selected from asciminib, imatinib, nilotinib, dasatinib, bosutinib, ponatinib and bafetinib are administered in temporal proximity (e.g., Compound A and the tyrosine-kinase inhibitor selected from asciminib, imatinib, nilotinib, dasatinib, bosutinib, ponatinib and bafetinib can be initially administered simultaneously). Accordingly, the present disclosure provides a method of treating or preventing cancer comprising administering Compound A and a tyrosine-kinase inhibitor selected from asciminib, imatinib, nilotinib, dasatinib, bosutinib, ponatinib and bafetinib in temporal proximity. In some embodiments, “temporal proximity” means that administration of one therapeutic agent occurs within a time period before or after the administration of another therapeutic agent, such that the therapeutic effect of the one therapeutic agent overlaps with the therapeutic effect of the another therapeutic agent. In some embodiments, the therapeutic effect of the one therapeutic agent completely overlaps with the therapeutic effect of the other therapeutic agent.

In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some embodiments, the disclosure provides a synergistic combination of Compound A and the tyrosine-kinase inhibitor selected from asciminib, imatinib, nilotinib, dasatinib, bosutinib, ponatinib and bafetinib, wherein Compound A and the tyrosine-kinase inhibitor selected from asciminib, imatinib, nilotinib, dasatinib, bosutinib, ponatinib and bafetinib come into contact with each other in the human body (e.g., only in the human body).

In some embodiments, Compound A and the tyrosine-kinase inhibitor selected from asciminib, imatinib, nilotinib, dasatinib, bosutinib, ponatinib and bafetinib are co-administration concurrently (e.g., simultaneously, essentially simultaneously or within the same treatment protocol) or sequentially.

In some embodiments, Compound A and the tyrosine-kinase inhibitor selected from asciminib, imatinib, nilotinib, dasatinib, bosutinib, ponatinib and bafetinib are co-administered in separate dosage forms. In some embodiments, Compound A and the tyrosine-kinase inhibitor selected from asciminib, imatinib, nilotinib, dasatinib, bosutinib, ponatinib and bafetinib are co-administered in combined dosage forms.

### Pharmaceutical Compositions and Formulations

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound of formula (I), (I-i), (IA), (IA-1), (IIA), (IIB), (IIE), or (Id), such as Compound A, intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, and without limitation, a composition of substantially pure(S) compound means that the composition contains no more than 15% or no more than 10% or no more than 5% or no more than 3% or no more than 1% of the (R) form of the compound.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21st ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described.

Compositions comprising a compound provided herein are also described. In one variation, the composition comprises a compound and a pharmaceutically acceptable carrier or excipient. In another variation, a composition of substantially pure compound is provided.

### BCR:ABL-1 Variants

In some embodiments, the BCR:ABL-1 protein is wild-type BCR:ABL-1 protein. In some embodiments, the BCR:ABL-1 protein contains a single amino acid mutation relative to wild-type BCR:ABL-1. In some embodiments, the BRC:ABL-1 protein contains multiple amino acid mutations relative to wild-type BCR:ABL-1.

In some embodiments, the BRC:ABL-1 protein contains a single amino acid mutation selected from F359V, F359C, F359I, H396R, E255V, T315I, A337V, A344P, P465S, T315M, and V468F. In some embodiments, the BCR:ABL-1 protein contains multiple amino acid mutations selected from (i) G250E and T315I, (ii) Y253H and T315I, (iii) E255V and T315I, (iv) H396R and T315I, (v) E255V and V299L, (vi) Y253H and F317L, (vii) A337V and T315I, (viii) A344P and T315I, (ix) P465S and T315I, and (x) V468F and T315I.

Wild-Type BCR:ABL-1, Isoform P00519
(SEQ ID NO: 1)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 F359V Single Mutant,
(SEQ ID NO: 2)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNVI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 F359C Single Mutant,
(SEQ ID NO: 3)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNCI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 F359I Single Mutant,
(SEQ ID NO: 4)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNII
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 H396R Single Mutant,
(SEQ ID NO: 5)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTARAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 E255V Single Mutant,
(SEQ ID NO: 6)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRENTLAELVHHHSTVADGLITTLHYPAPKRNKPTVYGVSPNYDKWEMERT
DITMKHKLGGGQYGVVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 T315I Single Mutant,
(SEQ ID NO: 7)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 A337V Single Mutant,
(SEQ ID NO: 8)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNVVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 A344P Single Mutant,
(SEQ ID NO: 9)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMPTQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 P465S Single Mutant,
(SEQ ID NO: 10)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCSEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 T315M Single Mutant,
(SEQ ID NO: 11)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIMEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 V468F Single Mutant,
(SEQ ID NO: 12)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKFYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 G250E/T315I Double Mutant,
(SEQ ID NO: 13)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGEGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 Y253H/T315I Double Mutant
(SEQ ID NO: 14)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQHGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 E255V/T315I, Double Mutant
(SEQ ID NO: 15)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGVVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 H396R/T315I, Double Mutant
(SEQ ID NO: 16)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRENTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTARAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 E255V/V299L, Double Mutant
(SEQ ID NO: 17)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRENTLAELVHHHSTVADGLITTLHYPAPKRNKPTVYGVSPNYDKWEMERT
DITMKHKLGGGQYGVVYEGVWKKYSLTVAVKTLKEDTMEVEEFLKEAAVMKEIKHPNLLQ
LLGVCTREPPFYIITEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 Y253H/F317L Double Mutant,
(SEQ ID NO: 18)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRENTLAELVHHHSTVADGLITTLHYPAPKRNKPTVYGVSPNYDKWEMERT
DITMKHKLGGGQHGEVYEGVWKKYSLTVAVKTLKEDTMEVEEFLKEAAVMKEIKHPNLVQ
LLGVCTREPPFYIITELMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 A337V/T315I, Double Mutant
(SEQ ID NO: 19)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNVVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 A344P/T315I, Double Mutant,
(SEQ ID NO: 20)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRENTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMPTQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 P465S/T315I, Double Mutant,
(SEQ ID NO: 21)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCSEKVYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR
BCR:ABL-1, Isoform P00519 V468F/T315I, Double Mutant,
(SEQ ID NO: 22)
MLEICLKLVGCKSKKGLSSSSSCYLEEALQRPVASDFEPQGLSEAARWNSKENLLAGPSE
NDPNLFVALYDFVASGDNTLSITKGEKLRVLGYNHNGEWCEAQTKNGQGWVPSNYITPVN
SLEKHSWYHGPVSRNAAEYLLSSGINGSFLVRESESSPGQRSISLRYEGRVYHYRINTAS
DGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQ
LLGVCTREPPFYIIIEFMTYGNLLDYLRECNRQEVNAVVLLYMATQISSAMEYLEKKNFI
HRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKFYELMRACWQWNP
SDRPSFAEIHQAFETMFQESSISDEVEKELGKQGVRGAVSTLLQAPELPTKTRTSRRAAE
HRDTTDVPEMPHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTPLDTADPAKSP
KPSNGAGVPNGALRESGGSGFRSPHLWKKSSTLTSSRLATGEEEGGGSSSKRFLRSCSAS
CVPHGAKDTEWRSVTLPRDLQSTGRQFDSSTFGGHKSEKPALPRKRAGENRSDQVTRGTV
TPPPRLVKKNEEAADEVFKDIMESSPGSSPPNLTPKPLRRQVTVAPASGLPHKEEAGKGS
ALGTPAAAEPVTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQPGEGLKKPVL
PATPKPQSAKPSGTPISPAPVPSTLPSASSALAGDQPSSTAFIPLISTRVSLRKTRQPPE
RIASGAITKGVVLDSTEALCLAISRNSEQMASHSAVLEAGKNLYTFCVSYVDSIQQMRNK
FAFREAINKLENNLRELQICPATAGSGPAATQDFSKLLSSVKEISDIVQR

### Methods of Use/Treatments

Compounds and compositions detailed herein, such as a pharmaceutical composition containing a compound provided herein, or a salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Compounds and compositions described herein may in some aspects be used in treatment of cancer. In some embodiments, the method of treating cancer in a subject in need thereof comprises administering to the subject a BCR:ABL1 inhibitor and a tyrosine kinase inhibitor (TKI).

In some embodiments, the method comprises administering to the subject a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib).

In some embodiments, the BCR:ABL1 inhibitor is Compound A, or a pharmaceutically acceptable salt thereof, and the tyrosine kinase inhibitor is imatinib. In some embodiments, the BCR:ABL1 inhibitor is Compound A, or a pharmaceutically acceptable salt thereof, and the tyrosine kinase inhibitor is dasatinib. In some embodiments, the BCR:ABL1 inhibitor is Compound A, or a pharmaceutically acceptable salt thereof, and the tyrosine kinase inhibitor is ponatinib.

Without being bound by theory, it is believed that the combination of a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib) in accordance with the methods described herein may effectively provide treatment as compared to monotherapies and thus reduce dose-dependent adverse effects that may accompany monotherapy treatment.

In one aspect, provided herein is a method of treating a cancer in a patient comprising administering to the patient a therapeutically effective amount of a compound or composition provided herein. In some embodiments, the cancer is melanoma, hereditary leiomyomatosis, renal cell carcinoma (HLRCC), or other solid tumors. In some embodiments, the cancer is lymphoma and leukemia. In some embodiments, the cancer is Chronic Myeloid Leukemia (CML).

Provided herein are methods of treating cancer in a patient (e.g., a human patient) in need thereof with a combination of a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), comprising administering a therapeutically effective amount of the BCR:ABL1 inhibitor, or a pharmaceutically acceptable salt thereof and a therapeutically effective amount of the tyrosine kinase inhibitor, wherein the cancer is melanoma, hereditary leiomyomatosis, renal cell carcinoma (HLRCC), or other solid tumors.

Provided herein are methods of treating cancer in a patient (e.g., a human patient) in need thereof with a combination of a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), comprising administering a therapeutically effective amount of the BCR:ABL1 inhibitor, or a pharmaceutically acceptable salt thereof and a therapeutically effective amount of the tyrosine kinase inhibitor, wherein the cancer is lymphoma and leukemia.

Provided herein are methods of treating cancer in a patient (e.g., a human patient) in need thereof with a combination of a BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), comprising administering a therapeutically effective amount of the BCR:ABL1 inhibitor and a therapeutically effective amount of the tyrosine kinase inhibitor, wherein the cancer is CML.

In some embodiments, the patient has had one or more prior therapies. In some embodiments, the cancer progressed during the therapy.

In some embodiments, the BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), are administered simultaneously. In some such embodiments, the BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), can be provided in a single pharmaceutical composition. In other embodiments, the BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib), are administered sequentially.

The BCR:ABL1 inhibitor (e.g., a compound of formula (I), (I-i), (IA), (IA-1), (IIA), (IIB), (IIE), or (Id), such as Compound A), or a pharmaceutically acceptable salt thereof and a tyrosine kinase inhibitor (e.g., imatinib, nilotinib, dasatinib, bosutinib, ponatinib and bafetinib) may be administered at doses that are typically administered when either agent is administered alone.

Alternatively, as a result of the likely synergy observed with the combination, the BCR:ABL1 inhibitor (e.g., a compound of formula (I), (I-i), (IA), (IA-1), (IIA), (IIB), (IIE), or (Id), such as Compound A), or a pharmaceutically acceptable salt thereof and/or a tyrosine kinase inhibitor (e.g., imatinib, nilotinib, dasatinib, bosutinib, ponatinib and bafetinib) may be administered at doses that are lower than doses when each agent is administered alone.

In some embodiments, the BCR:ABL1 inhibitor (e.g., a compound of formula (I), (I-i), (IA), (IA-1), (IIA), (IIB), (IIE), or (Id), such as Compound A), or a pharmaceutically acceptable salt thereof, used in accordance with the method described herein, can be administered to an individual a once daily dose for a first period of time, followed by a second period of time in which administration of the compound may be discontinued, wherein the tyrosine kinase inhibitor (e.g., imatinib, nilotinib, dasatinib, bosutinib, ponatinib and bafetinib) may be maintained during both the first and the second period of time.

In one aspect, provided herein is a method of inhibiting tyrosine kinase enzymatic activity of a protein selected from Abelson protein (ABL1), Abelson-related protein (ABL2), or a chimeric protein BCR-ABL1, comprising contacting an effective amount of Compound A in combination with an effective amount of a tyrosine-kinase inhibitor, with the protein. In one embodiment, provided herein is a method of inhibiting tyrosine kinase enzymatic activity of Abelson protein (ABL1) comprising contacting an effective amount of Compound A in combination with an effective amount of a tyrosine-kinase inhibitor to ABL1. In another embodiment, provided herein is a method of inhibiting tyrosine kinase enzymatic activity of Abelson-related protein (ABL2) comprising contacting an effective amount of Compound A in combination with an effective amount of a tyrosine-kinase inhibitor to ABL2. In a further embodiment, provided herein is a method of inhibiting tyrosine kinase enzymatic activity of a chimeric protein BCR-ABL1 comprising contacting an effective amount of Compound A in combination with an effective amount of a tyrosine-kinase inhibitor to the chimeric protein.

In one aspect, provided herein is a method of treating a disease in a patient in need thereof, comprising administering to the patient a therapeutically effective amount of Compound A or a salt thereof in combination with a therapeutically amount of a tyrosine-kinase inhibitor.

The compounds or salts thereof, the combinations, and compositions described herein are believed to be effective for treating a variety of diseases and disorders. In some embodiments, a compound or salt thereof, a combination, and a composition described herein may be used in a method of treating a disease mediated by ABL1, ABL2, and/or BCR-ABL1.

In one aspect, provided herein is a method of treating a disease, wherein modulation of BCR-ABL1 activity prevents, inhibits, or ameliorates the pathology and/or symptomology of the disease, in a patient, comprising administering to the patient a therapeutically effective amount of Compound A in combination with a therapeutically effective amount of a tyrosine-kinase inhibitor. In one embodiment, provided herein is a method of treating a disease, wherein modulation of BCR-ABL1 activity prevents the pathology and/or symptomology of the disease, in a patient, comprising administering to the patient a therapeutically effective amount of Compound A in combination with a therapeutically effective amount of a tyrosine-kinase inhibitor. In one embodiment, provided herein is a method of treating a disease, wherein modulation of BCR-ABL1 activity inhibits the pathology and/or symptomology of the disease, in a patient, comprising administering to the patient a therapeutically effective amount of Compound A in combination with a therapeutically effective amount of a tyrosine-kinase inhibitor. In one embodiment, provided herein is a method of treating a disease, wherein modulation of BCR-ABL1 activity ameliorates the pathology and/or symptomology of the disease, in a patient, comprising administering to the patient a therapeutically effective amount of Compound A in combination with a therapeutically effective amount of a tyrosine-kinase inhibitor.

In some embodiments, the disease is leukemia. In some embodiments, the leukemia is chronic myeloid leukemia (CML), acute myeloid leukemia (AML), or acute lymphoblastic leukemia (ALL). In some embodiments, the leukemia is chronic myeloid leukemia (CML).

In one aspect, provided herein is a method of treating leukemia in a patient comprising administering to the patient a therapeutically effective amount of Compound A in combination with a therapeutically effective amount of a tyrosine-kinase inhibitor. In one aspect, provided herein is a method of treating leukemia in a patient comprising administering to the patient a therapeutically effective amount of Compound A in combination with a therapeutically effective amount of a tyrosine-kinase inhibitor, wherein the leukemia is chronic myeloid leukemia (CML), acute myeloid leukemia (AML), or acute lymphoblastic leukemia (ALL).

In some embodiments, the leukemia treated herein is CML or ALL, and the method comprises administering a therapeutically effective amount of Compound A in combination with a therapeutically effective amount of a tyrosine-kinase inhibitor selected from asciminib, imatinib, nilotinib, dasatinib, bosutinib, ponatinib and bafetinib.

In some embodiments, the leukemia is resistant to treatment. In some embodiments, the leukemia is resistant to treatment with asciminib, imatinib, nilotinib, dasatinib, bosutinib, ponatinib and/or bafetinib. In some embodiments, the CML is resistant to standard-of-care treatment such as treatment with one or more of asciminib, imatinib, nilotinib, dasatinib, bosutinib, ponatinib and bafetinib. In some embodiments, the leukemia progressed during a prior treatment. In some embodiments the prior treatment comprised administration of asciminib, imatinib, nilotinib, dasatinib, bosutinib, ponatinib and/or bafetinib.

In some embodiments, the AML is secondary AML, which develops after myelodysplastic syndromes (MDS) or myeloproliferative neo-plasms (MPN).

In another aspect is provided a method of delaying the onset and/or development of a disease or disorder that is mediated by BCR-ABL1 activity in a patient (such as a human) who is at risk for developing the disease or disorder. It is appreciated that delayed development may encompass prevention in the event the individual or patient does not develop the disease or disorder. In one aspect, an individual or patient at risk of developing a disease or disorder that is mediated by BCR-ABL1 activity has one or more risk factors for developing the disease or disorder, such as a family history of an individual or patient having the disease or disorder, or having an underlying genetic condition that is associated with an increased likelihood of developing the disease or disorder.

In one aspect, provided herein is a method of delaying the onset and/or development of leukemia in a patient in need thereof, comprising administering to the patient a therapeutically effective amount of Compound A in combination with a therapeutically effective amount of a tyrosine-kinase inhibitor. In one variation, provided herein is a method of delaying the onset and/or development of CML in a patient in need thereof, comprising administering to the patient a therapeutically effective amount of Compound A in combination with a therapeutically effective amount of a tyrosine-kinase inhibitor. In one variation, provided herein is a method of delaying the onset and/or development of AML in a patient in need thereof, comprising administering to the patient a therapeutically effective amount of Compound A in combination with a therapeutically effective amount of a tyrosine-kinase inhibitor. In one variation, provided herein is a method of delaying the onset and/or development of ALL in a patient in need thereof, comprising administering to the patient a therapeutically effective amount of Compound A in combination with a therapeutically effective amount of a tyrosine-kinase inhibitor.

Methods of treating a disease mediated by BCR-ABL1, such as various leukemias and the like, are well known to the skilled artisan and can be adapted to treating such a disease with a combination comprising Compound A and a tyrosine-kinase inhibitor.

In some embodiments, the patient is a mammal. In some embodiments, the patient is a primate, dog, cat, rabbit, or rodent. In some embodiments, the patient is a primate. In some embodiments, the patient is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old. In some embodiments, the patient has a genetic condition that is associated with an increased likelihood of developing the disease, such as the leukemia. In some embodiments, the patient has a mutation in the ABL1 and/or ABL2 gene. In some embodiments, the patient is Philadelphia chromosome positive.

A combination, compound or composition provided herein may be administered to a patient in accordance with an effective dosing regimen for a desired period of time or duration, such as at least about one month, at least about 2 months, at least about 3 months, at least about 6 months, or at least about 12 months or longer, which in some variations may be for the duration of the patient's life. In one variation, the combination, compound or composition is administered on a daily or intermittent schedule. The combination, compound or composition can be administered to an patient continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent (e.g., once daily dosing for 7 days followed by no doses for 7 days, repeated for any 14 day time period, such as about 2 months, about 4 months, about 6 months or more). Any of the dosing frequencies can employ any of the combinations, compounds or compositions described herein together with any of the dosages described herein.

The combinations, compounds or compositions provided herein may be administered to a patient via various routes, including, e.g., intravenous, intramuscular, subcutaneous, oral and transdermal.

The dose of a compound administered to a patient may vary with the particular compound or salt thereof, the method of administration, and the particular disease. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g in a day, or about 7 mg to 350 mg in a day, or about 350 mg to 1.75 g in a day, or about 1.75 to 7 g in a day.

Also provided herein are uses of a combination, compound or composition described herein, in the manufacture of a medicament. In some embodiments, the manufacture of a medicament is for the treatment of a disease described herein. In some embodiments, the manufacture of a medicament is for the treatment of a disease mediated by ABL1, ABL2, and/or BCR-ABL1.

In some embodiments, the methods disclosed herein further comprise a step of determining the DNA sequence of the BCR:ABL1 gene expressed by the subject.

In some embodiments, the methods disclosed herein further comprise the step of determining the amino acid sequence of the BCR:ABL1 protein expressed by the subject.

### Dosing and Treatment Regimens

In some embodiments, the combination comprises administering Compound A and a tyrosine-kinase inhibitor selected from asciminib, imatinib, nilotinib, dasatinib, bosutinib, ponatinib and bafetinib at a ratio from about 1:10 to about 10:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:9 to about 9:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:8 to about 8:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:7 to about 7:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:6 to about 6:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:5 to about 5:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:4 to about 4:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:3 to about 3:1.

In some embodiments, the combination comprises administering Compound A and a tyrosine-kinase inhibitor selected from asciminib, imatinib, nilotinib, dasatinib, bosutinib, ponatinib and bafetinib at a ratio from about 1:2 to about 2:1.

In some embodiments, the combination comprises administering Compound A and a tyrosine-kinase inhibitor selected from asciminib, imatinib, nilotinib, dasatinib, bosutinib, ponatinib and bafetinib at a ratio of about 1:1, about 1:2, about 1:3, about 1:4, about 1:5, about 2:1, about 2:3, about 2:5, about 3:1, about 3:2, about 3:4, about 3:5, about 4:1, about 4:3, or about 4:5.

In some embodiments, the combination comprising Compound A and imatinib is synergistic in a K562 cell line over a Ku812 cell line.

In some embodiments, the combination comprising Compound A and dasatinib is synergistic in a K562 cell line over a Ku812 cell line.

### Articles of Manufacture and Kits

The present disclosure further provides articles of manufacture comprising a combination, compound or composition, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises a combination, compound or composition described herein. The kits may employ any of the combination, compound or composition disclosed herein. In some embodiments, the kit employs a BCR:ABL1 inhibitor (e.g., a compound of formula (I), (I-i), (IA), (IA-1), (IIA), (IIB), (IIE), or (Id), such as Compound A, or a pharmaceutically acceptable salt thereof) and a tyrosine kinase inhibitor (e.g., imatinib, nilotinib, dasatinib, bosutinib, ponatinib and bafetinib) described herein. The kits may be used for any one or more of the uses described herein, and, accordingly, may contain instructions for the treatment as described herein.

Kits generally comprise suitable packaging. The kits may comprise one or more containers comprising any combination described herein, any compound described herein or a pharmaceutically acceptable salt thereof. Each component can be packaged in separate containers or some components can be combined in one container where cross-reactivity and shelf life permit. In some embodiments, the kit includes a container comprising a BCR:ABL1 inhibitor (e.g., a compound of formula (I), (I-i), (IA), (IA-1), (IIA), (IIB), (IIE), or (Id), such as Compound A, or a pharmaceutically acceptable salt thereof) and a tyrosine kinase inhibitor (e.g., imatinib, nilotinib, dasatinib, bosutinib, ponatinib and bafetinib). In other embodiments, the kit includes a first container comprising a BCR:ABL1 inhibitor (e.g., a compound of formula (I), (I-i), (IA), (IA-1), (IIA), (IIB), (IIE), or (Id), such as Compound A, or a pharmaceutically acceptable salt thereof) and a second container comprising a tyrosine kinase inhibitor (e.g., imatinib, nilotinib, dasatinib, bosutinib, ponatinib and bafetinib).

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of a patient for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

The kits may be used for any one or more of the uses described herein, and, accordingly, may contain instructions for the treatment of any disease or described herein, for example for the treatment of cancer.

### Exemplary Embodiments

P-1. A method of treating cancer in a subject in need thereof, comprising administering a BCR:ABL1 inhibitor in combination with a tyrosine-kinase inhibitor, wherein the BCR:ABL1 inhibitor is a compound of formula (I):

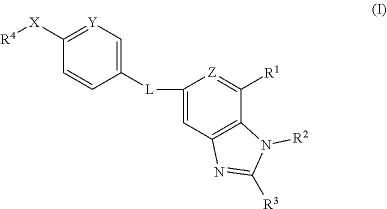

or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷,
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-.
P-2. The method of embodiment P-1, wherein the BCR:ABL1 inhibitor is a compound is of formula (IA-1):

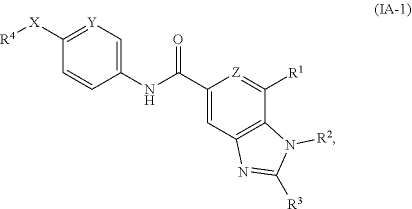

or a pharmaceutically acceptable salt thereof.
P-3. The method of any one of the preceding embodiments, wherein in the BCR:ABL1 inhibitor is (R)—N-(4-(chlorodifluoromethoxy)phenyl)-2-(difluoromethyl)-1-(1-hydroxypropan-2-yl)-7-(pyrimidin-5-yl)-1H-benzo[d]imidazole-5-carboxamide (Compound A):

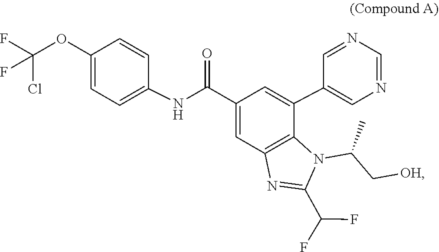 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof, in combination with a tyrosine-kinase inhibitor, for the treatment of cancer in a subject in need thereof.
P-4. The method of any one of the preceding embodiments, wherein the tyrosine-kinase inhibitor is selected from imatinib, nilotinib, dasatinib, bosutinib, ponatinib and bafetinib.
P-5. The method of any one of the preceding embodiments, wherein the cancer is lymphoma or leukemia.
P-6. A method of inhibiting tyrosine kinase enzymatic activity of a protein selected from the group consisting of Abelson protein (ABL1), Abelson-related protein (ABL2), and a chimeric protein BCR-ABL1, comprising contacting an effective amount of Compound A:

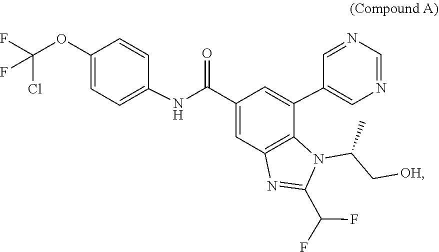 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof in combination with an effective amount of atyrosine-kinase inhibitor, with the protein.
P-7. A method of treating a disease, wherein modulation of BCR-ABL1 activity prevents, inhibits, or ameliorates the pathology and/or symptomology of the disease, in a patient, comprising administering to the patient a therapeutically effective amount of Compound A:

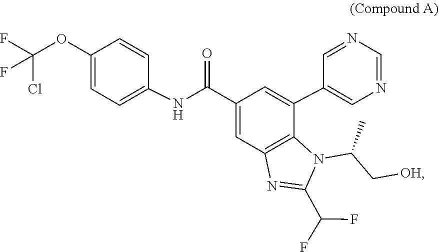 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof in combination with a therapeutically effective amount of a tyrosine-kinase inhibitor.
P-8. A method of treating leukemia in a patient comprising administering to the patient a therapeutically effective amount of Compound A:

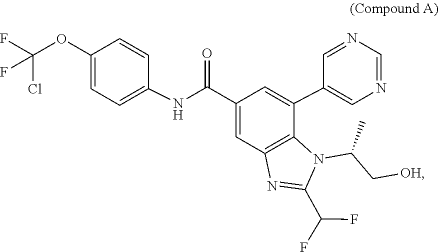 [SMILES: C[C@H](CO)n1c(C(F)F)nc2cc(C(=O)Nc3ccc(OC(F)(F)Cl)cc3)cc(-c3cncnc3)c21]

or a pharmaceutically acceptable salt thereof in combination with a therapeutically effective amount of a tyrosine-kinase inhibitor, wherein the leukemia is chronic myeloid leukemia (CML), acute myeloid leukemia (AML), or acute lymphoblastic leukemia (ALL).
P-9. The method of any one of the preceding embodiments, wherein the method comprises administering Compound A and a tyrosine-kinase inhibitor selected from imatinib, nilotinib, dasatinib, bosutinib, ponatinib and bafetinib at a ratio of about 1:1, about 1:2, about 1:3, about 1:4, about 1:5, about 2:1, about 2:3, about 2:5, about 3:1, about 3:2, about 3:4, about 3:5, about 4:1, about 4:3, or about 4:5.
P-10. Use of Compound A, or a pharmaceutically acceptable salt thereof, in combination with a tyrosine-kinase inhibitor, for the preparation of a medicament for the treatment of cancer in a subject in need thereof.
P-11. A combination comprising a BCR:ABL1 inhibitor and a tyrosine-kinase inhibitor, wherein the BCR:ABL1 inhibitor is a compound of formula (I):

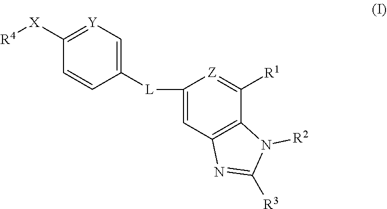

or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷;
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-;
and at least one pharmaceutically acceptable excipient.
P-12. The combination of embodiment P-11, wherein the combination is used for treating leukemia in a patient comprising administering to the patient a therapeutically effective amount of the BCR:ABL1 inhibitor and a therapeutically effective amount of the tyrosine-kinase inhibitor, wherein the leukemia is chronic myeloid leukemia (CML), acute myeloid leukemia (AML), or acute lymphoblastic leukemia (ALL).
P-13. The combination for use according to embodiment P-12, wherein the leukemia is CML or ALL, and the tyrosine-kinase inhibitor is selected from the group selected from imatinib, nilotinib, dasatinib, flumatinib, bosutinib, ponatinib and bafetinib.
P-14. The combination for use according to any one of the preceding embodiments, wherein the CML is resistant to standard-of-care treatment.
P-15. The combination for use according to any one of the preceding embodiments, wherein the CML is resistant to treatment with one or more of imatinib, ponatinib, nilotinib, and dasatinib.
P-16. The combination for use according to any one of the preceding embodiments, wherein the AML is secondary AML, which develops after myelodysplastic syndromes (MDS) or myeloproliferative neo-plasms (MPN).

### EXAMPLE

### Example 1—Potency, Selectivity, and Ability of Compound A to Work Synergistically with Active Site TKIs

### Background

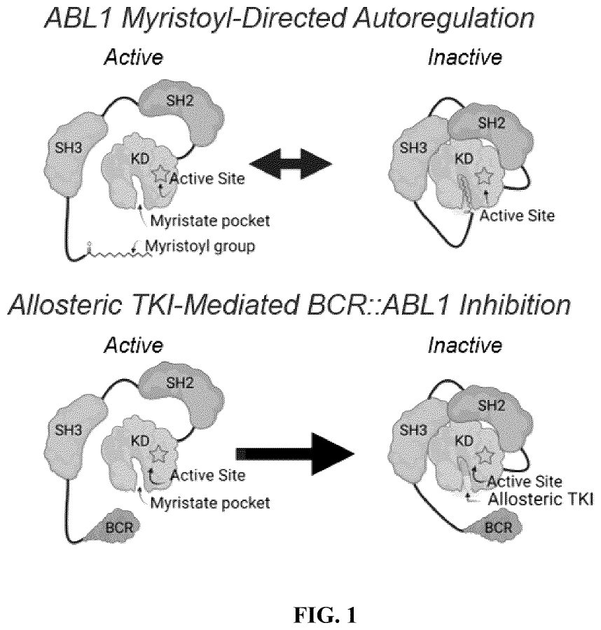

Chronic Myeloid Leukemia (CML) is a myeloproliferative disorder characterized by a reciprocal translocation between chromosomes 9 and 22, leading to the loss of myristoyl-directed autoregulation and constitutive activation of the BCR:ABL1 oncoprotein (FIG. 1).

Compound A retains activity against the BCR::ABL1T315I resistance mutation, which confers resistance to all approved active site inhibitors except for ponatinib.

### Objective

Although tyrosine kinase inhibitors (TKIs) targeting the ATP-binding site of the BCR::ABL1 oncoprotein are effective therapeutics for chronic myeloid leukemia (CML), patients often develop drug resistance due to ATP-site mutations that inhibit drug binding. Compound A acts allosterically to specifically target the ABL Myristoyl Pocket (STAMP) of BCR:ABL1, resulting in its inhibition. Compound A is designed to circumvent the resistance of active site mutations, with potential for synergistic combination with active site TKIs.

### Methods

The ABL1 kinase domain (amino acid residues 64-515) was expressed and purified via affinity chromatography from SH9 cells. Activity was validated using a microfluidic mobility shift assay.

Substrate phosphorylation assays were used to assess the effect of Compound A on BCR::ABL1 and other kinases. The ability of Compound A to inhibit the proliferation of wild type and mutant CML cell lines was assessed using CellTiter-Glo® or ATPlite 1Step™ (PerkinElmer) solution. Synergy between Compound A and active site TKIs were assessed using both fixed molar combinations and expanded combination matrices, with cell viability measured using CellTiter-Glo® and interactions quantified using the Bliss, HSA, and Loewe models.

### Results

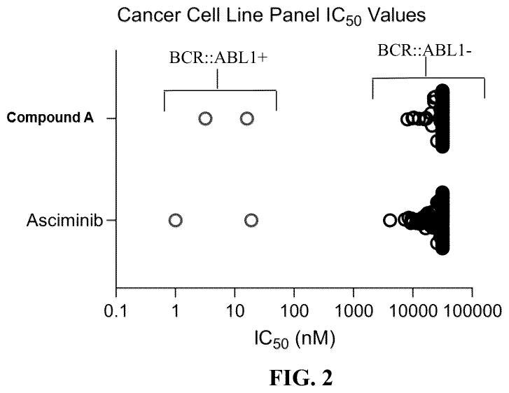

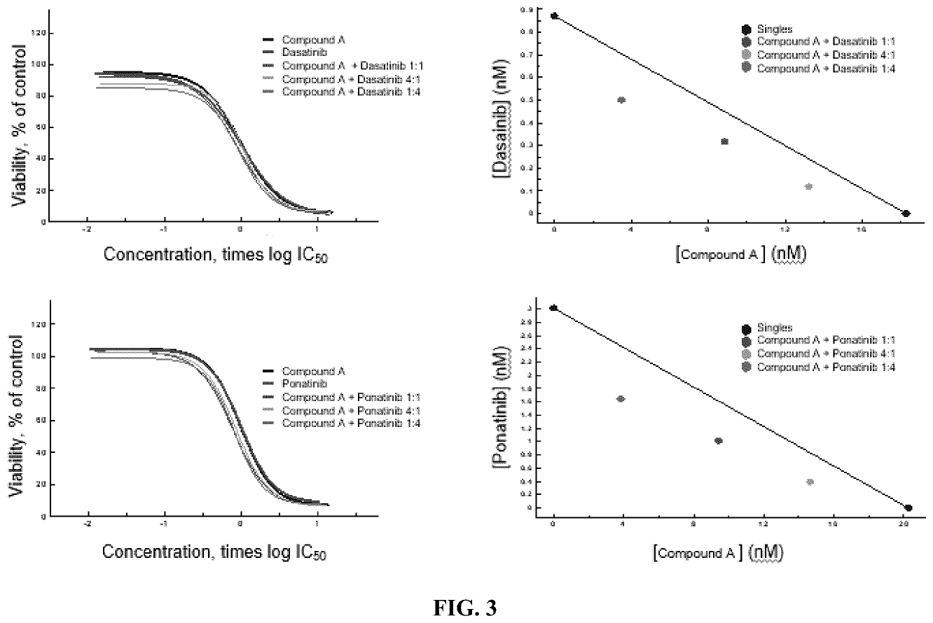

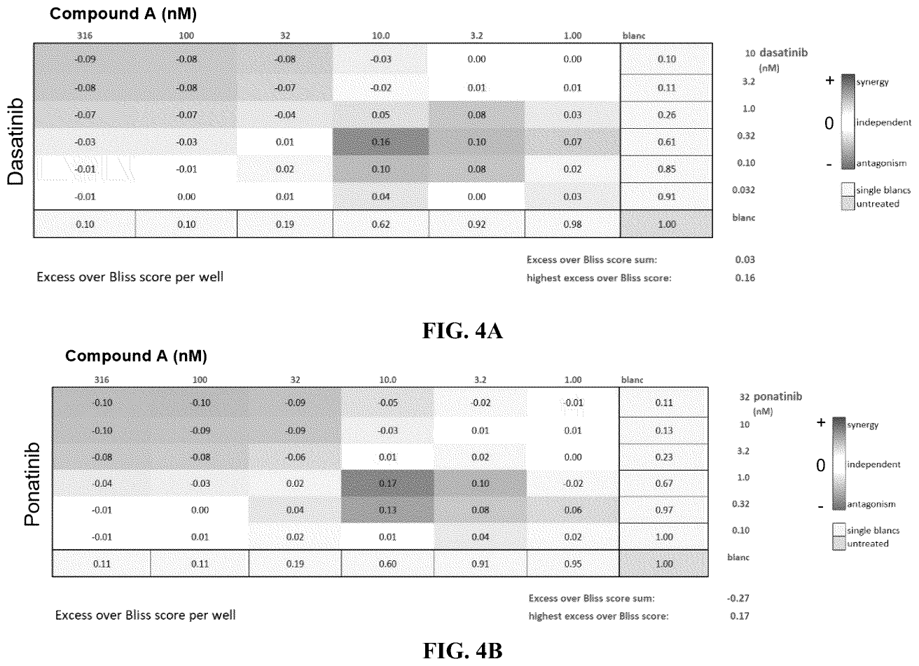

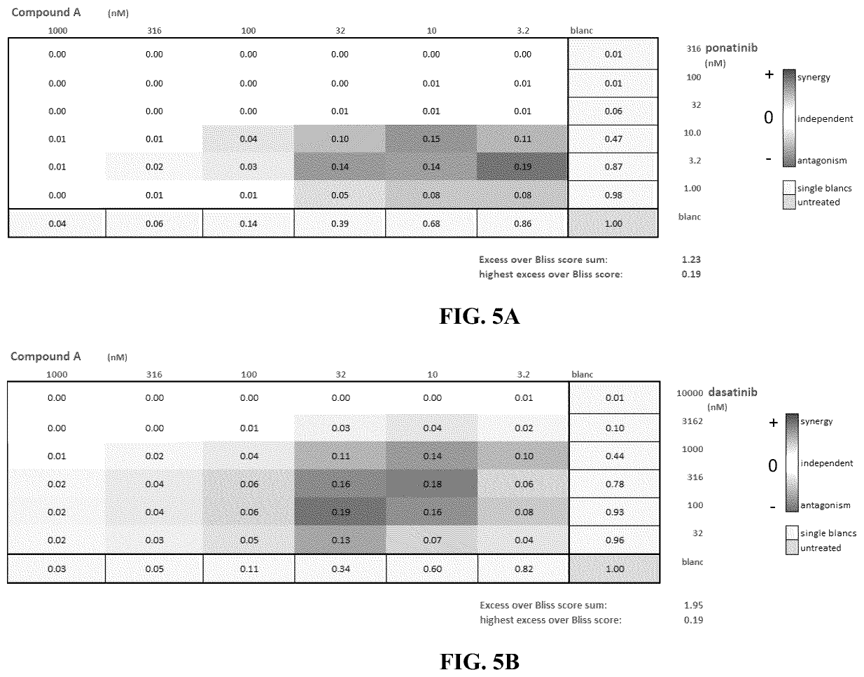

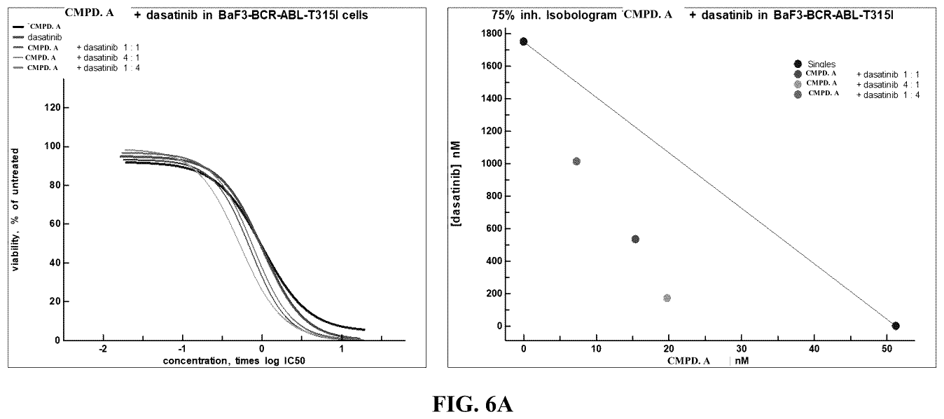

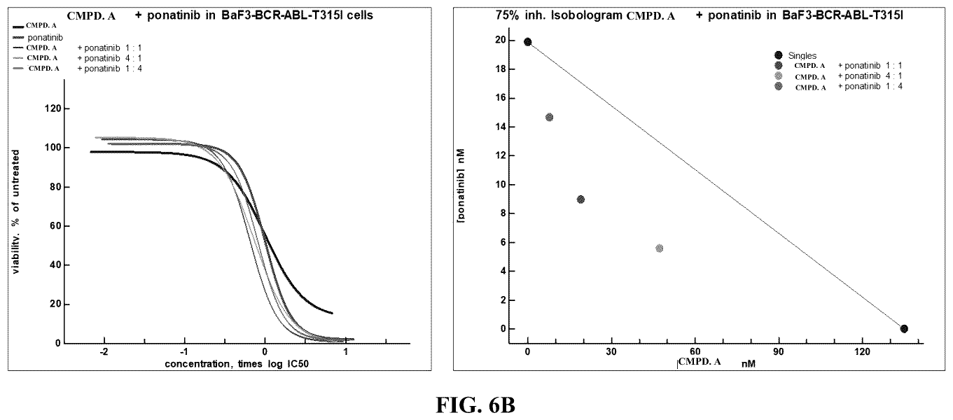

With respect to selectivity, in an in vitro kinase panel, Compound A did not inhibit any kinase by >50% at 1 μM, including full-length ABL1. Compound A was highly potent and selective against only BCR::ABL1+ cell lines in cancer cell line panel, and was more selective than asciminib (FIG. 2). In vitro combination studies revealed that Compound A works synergistically with multiple TKIs in the K562 and BaF3BCR-ABL-T315I cell lines, and at least additively in the Ku812 cell line (FIGS. 3-6B and Tables 1-4). As assessed using the BLISS model, synergy was strongest at and below the IC₅₀ of each single compound.

TABLE 1
Summary of the synergy as quantified by curve shift and
isobologram analyses

| | Combination | Cell | |
|---|---|---|---|
| Compound | Partner | Line | Summary of Observations |
| Compound | Imatinib | K562 | Synergy at IC₇₅ and IC₉₀ |
| A | | Ku812 | Additivity |
| | Dasatinib | K562 | Synergy at all molar |
| | | | combinations |
| | | Ku812 | Additivity |
| | Ponatinib | K562 | Synergy at all molar |
| | | | combinations |
| | | Ku812 | Additivity |
| Asciminib | Imatinib | K562 | Synergy at IC₇₅ and IC₉₀ |
| | | Ku812 | Additivity |
| | Dasatinib | K562 | Synergy at IC90 |
| | | Ku812 | Additivity |
| | Ponatinib | K562 | Synergy at IC₇₅ and IC₉₀ |
| | | Ku812 | Additivity |

TABLE 2
Summarized BLISS synergy data of Compound A in combination with
imatinib, dasatinib or ponatinib

| | | | | Strongest |
|---|---|---|---|---|
| | Combination | Cell | Summary of | BLISS |
| Compound | Partner | Line | Observations | score |
| Compound A | Imatinib | K562 | BLISS synergy | 0.12 |
| | | | observed at and | |
| | | | below IC₅₀ of single | |
| | | | compounds | |
| | | Ku812 | Weak synergy observed | 0.05 |
| | Dasatinib | K562 | BLISS synergy | 0.16 |
| | | | observed at and | |
| | | | below IC₅₀ of | |
| | | | single compounds | |
| | | Ku812 | Weak synergy observed | 0.08 |
| | Ponatinib | K562 | BLISS synergy | 0.17 |
| | | | observed at and | |
| | | | below IC₅₀ of | |
| | | | single compounds | |
| | | Ku812 | Weak synergy observed | 0.07 |

TABLE 3
Summarized BLISS synergy data of Compound A in combination
with imatinib, dasatinib or ponatinib

| Compound | Compound | | Inc. | Excess over | Highest excess | |
|---|---|---|---|---|---|---|
| 1 | 2 | cell line | time | Bliss score sum | over Bliss score | FIG. |
| Asciminib | dasatinib | BaF3-BCR- | 72 | 3.19 | 0.27 | — |
| | | ABL-T315I | hours | | | |
| Asciminib | flumatinib | BaF3-BCR- | 72 | 0.81 | 0.15 | — |
| | | ABL-T315I | hours | | | |
| Asciminib | imatinib | BaF3-BCR- | 72 | 1.29 | 0.15 | — |
| | | ABL-T315I | hours | | | |
| Asciminib | nilotinib | BaF3-BCR- | 72 | 0.36 | 0.05 | — |
| | | ABL-T315I | hours | | | |
| Asciminib | ponatinib | BaF3-BCR- | 72 | 0.83 | 0.14 | — |
| | | ABL-T315I | hours | | | |
| Compound | dasatinib | BaF3-BCR- | 72 | 1.95 | 0.19 | 5b |
| A | | ABL-T315I | hours | | | |
| Compound | flumatinib | BaF3-BCR- | 72 | 0.68 | 0.14 | — |
| A | | ABL-T315I | hours | | | |
| Compound | imatinib | BaF3-BCR- | 72 | 0.89 | 0.14 | — |
| A | | ABL-T315I | hours | | | |
| Compound | nilotinib | BaF3-BCR- | 72 | 0.14 | 0.06 | — |
| A | | ABL-T315I | hours | | | |
| Compound | ponatinib | BaF3-BCR- | 72 | 1.23 | 0.19 | 5a |
| A | | ABL-T315I | hours | | | |

TABLE 4
Summary of the synergy as quantified by curve shift and
isobologram analyses

| | | | Incubation | | |
|---|---|---|---|---|---|
| Compound | Reference | Cell line | time | Conclusion | Figure |
| Ascminib | dasatinib | BaF3BCR- | 72 hours | synergy | - |
| | | ABL-T315I | | | |
| Ascminib | flumatinib | BaF3BCR- | 72 hours | no synergy | - |
| | | ABL-T315I | | | |
| Ascminib | imatinib | BaF3BCR- | 72 hours | synergy at | - |
| | | ABL- | | 75% and | |
| | | T315I | | 90% | |
| | | | | inhibition | |
| | | | | (CI75 | |
| | | | | and CI90) | |
| Ascminib | nilotinib | BaF3BCR- | 72 hours | no synergy | - |
| | | ABL-T315I | | | |
| Ascminib | ponatinib | BaF3BCR- | 72 hours | synergy at | - |
| | | ABL-T315I | | ratio 1:1 | |
| Compound A | dasatinib | BaF3BCR- | 72 hours | synergy | 6A |
| | | ABL-T315I | | | |
| Compound A | flumatinib | BaF3BCR- | 72 hours | no synergy | - |
| | | ABL-T315I | | | |
| Compound A | imatinib | BaF3BCR- | 72 hours | no synergy | - |
| | | ABL-T315I | | | |
| Compound A | nilotinib | BaF3BCR- | 72 hours | no synergy | - |
| | | ABL-T315I | | | |
| Compound A | ponatinib | BaF3BCR- | 72 hours | synergy | 6B |
| | | ABL-T315I | | | |

### Conclusions

Compound A is a potent and selective allosteric inhibitor of BCR::ABL1 in cell-free and cell-based assays and screens, with comparable potency and synergy profiles to that of asciminib while potentially being more selective. Compound A retains activity against the T315I gatekeeper mutation, which confers resistance to all approved active site TKIs except for ponatinib.

This study demonstrated that Compound A, a BCR::ABL1 allosteric inhibitor, is potent against mutations resistant to active site tyrosine kinase inhibitors (TKIs) and acts synergistically with TKIs in BCR::ABL1+ cancer cell lines.

### Example 2: Determine the Efficacy of Compound A (ABT4) Alone and in Combination with Dasatinib (ABT6) and Ponatinib (ABT7) in Ba/F3-T3150 Murine CML in BALB/c Nude Female Mice

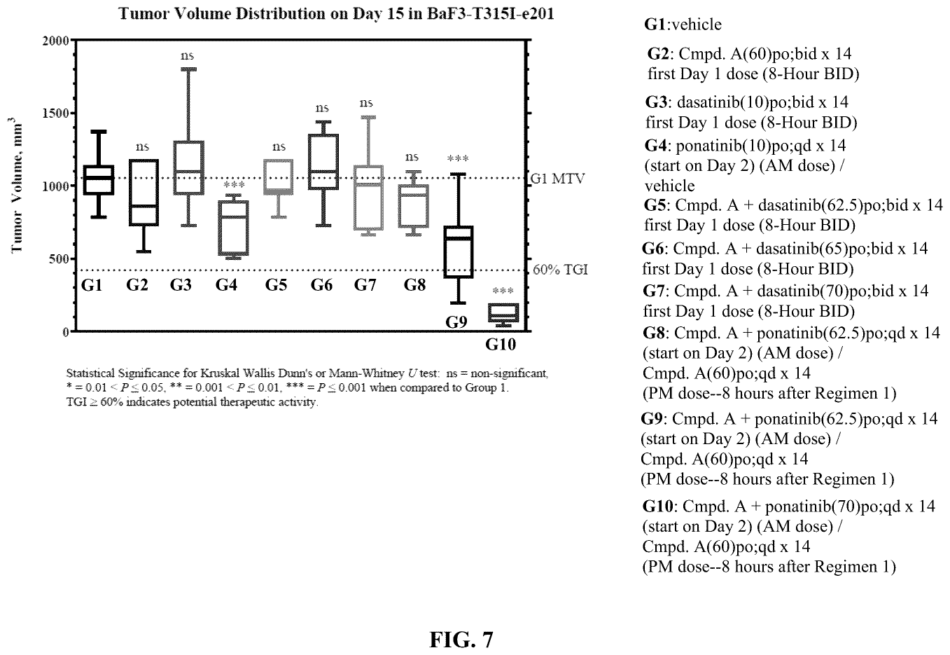

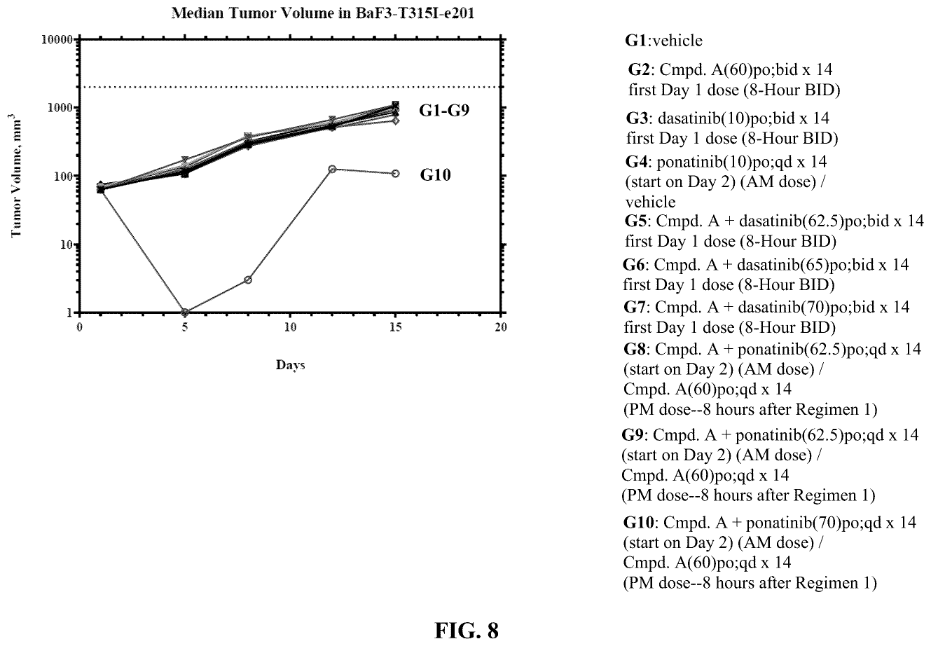

Example 2 describes a study conducted an in vivo study demonstrating that Compound A, when combined with ponatinib, shows a dose-dependent reduction in tumor burden in a BCR::ABL1-T315I CML model. Results are summarized in FIGS. 7-8.

### Procedures

Set up CR 245 female BALB/c nude mice with 2×10⁶ BaF3-T315I tumor cells in 0% Matrigel sc in flank.

Cell Injection Volume is 0.1 mL/mouse.

Age at Start Date: 7 to 10 weeks.

Performed a pair match when tumors reach an average size of 60-90 mm³ and began treatment

Body Weight: qd×5 then biwk to end

Caliper Measurement: biwk to end

Any individual animal with a single observation of >than 30% body weight loss or three consecutive measurements of >25% body weight loss was euthanized.

Any group with a mean body weight loss of >20% or >10% mortality stopped dosing. The group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery are allowed on a case-by-case basis.

Endpoint. Animals were monitored as a group. The endpoint of the experiment was a mean tumor weight in Control Group of 2000 mm³ or 15 days, whichever came first. When the endpoint was reached, all the animals were euthanized.

Groups of animals were dosed with compounds described below:

TABLE 2-1
Drugs and Treatment:

| | | Agent | Active dose | Route | Schedule |
|---|---|---|---|---|---|
| # | 0 | vehicle | na | po | bid × 14 first day 1 dose (8-Hour BID) |
| | 0 | ABT4 | 60 mg/kg | po | bid × 14 first day 1 dose (8-Hour BID) |
| | 0 | dasatinib | 10 mg/kg | po | bid × 14 first day 1 dose (8-Hour BID) |
| | 0 | ponatinib | 10 mg/kg | po // po | qd × 14 (start on day 2) (AM dose) // |
| | | // | | | qd × 14 (PM dose—8 hours after Regimen 1) |
| | | vehicle | | | |
| | 0 | ABT6 | 62.5 mg/kg | po | bid × 14 first day 1 dose (8-Hour BID) |
| | 0 | ABT6 | 65 mg/kg | po | bid × 14 first day 1 dose (8-Hour BID) |
| | 0 | ABT6 | 70 mg/kg | po | bid × 14 first day 1 dose (8-Hour BID) |
| | 0 | ABT7// | 62.5 mg/kg // | po // po | qd × 14 (start on day 2) (AM dose) // |
| | | ABT4 | 60 mg/kg | | qd × 14 (PM dose—8 hours after Regimen 1) |
| | 0 | ABT7// | 65 mg/kg // | po // po | qd × 14 (start on day 2) (AM dose) // |
| | | ABT4 | 60 mg/kg | | qd × 14 (PM dose—8 hours after Regimen 1) |
| 0 | 0 | ABT7// | 70 mg/kg // | po // po | qd × 14 (start on day 2) (AM dose) // |
| | | ABT4 | 60 mg/kg | | qd × 14 (PM dose—8 hours after Regimen 1) |
| 1 | | ABT4 | 60 mg/kg | po | bid × 7 first day 1 dose (8-Hour BID) |
| 2 | | dasatinib | 10 mg/kg | po | bid × 7 first day 1 dose (8-Hour BID) |
| 3 | | ponatinib // | 10 mg/kg // | po // po | qd × 7 (start on day 2) (AM dose) // |
| | | vehicle | | | qd × 7 (PM dose—8 hours after Regimen 1) |
| 4 | | ABT6 | 62.5 mg/kg | po | bid × 7 first day 1 dose (8-Hour BID) |
| 5 | | ABT6 | 65 mg/kg | po | bid × 7 first day 1 dose (8-Hour BID) |
| 6 | | ABT6 | 70 mg/kg | po | bid × 7 first day 1 dose (8-Hour BID) |
| 7 | | ABT7// | 62.5 mg/kg // | po // po | qd × 7 (start on day 2) (AM dose) // |
| | | ABT4 | 60 mg/kg | | qd × 7 (PM dose—8 hours after Regimen 1) |
| 8 | | ABT7// | 65 mg/kg // | po // po | qd × 7 (start on day 2) (AM dose) // |
| | | ABT4 | 60 mg/kg | | qd × 7 (PM dose—8 hours after Regimen 1) |
| 9 | | ABT7// | 70 mg/kg /! | po // po | qd × 7 (start on day 2) (AM dose) // |
| | | ABT4 | 60 mg/kg | | qd × 7 (PM dose—8 hours after Regimen 1) |

ABT6 are co-formulated agents—dosage clarification below.

| | Active | | | |
|---|---|---|---|---|
| | Dosage | Compound A | dasatinib | ponatinib |
| ABT6 | 62.5 | 60 | 2.5 | |
| ABT6 | 65 | 60 | 5 | |
| ABT6 | 70 | 60 | 10 | |
| ABT7 | 62.5 | 60 | | 2.5 |
| ABT7 | 65 | 60 | | 5 |
| ABT7 | 70 | 60 | | 10 |

#-Control Group

### Example 3: Screening of Compound a Against BCR:ABL1 Mutants

### Cell Proliferation Assays for an Expanded Panel of BCR-ABL1 Single and Compound Mutant Cell Lines

Compound A will be screened against Ba/F3 cell lines expressing wild-type BCR-ABL1 (either in the pSRalpha or pMIG expression vector) or a panel of single or compound BCR-ABL1 mutations. BCR-ABL1 single mutant lines will include an array of clinically relevant kinase domain mutations as well as select mutants of the myristoylation pocket which confer resistance to asciminib; BCR-ABL1 compound mutants screened will include both T315I-inclusive and non-T315I compound mutations. Parental Ba/F3 cells will also be tested as a control for on-target selectivity. Briefly, Ba/F3 BCR-ABL1 cells lines will be resuspended in fresh culture media (with 10% FBS) and plated in 384-well plates in the presence of graded concentrations of Compound A. Plates will be cultured for 3 days and subjected to MTS-based colorimetric viability assay. Absorbance data will be blanked, normalized to untreated controls, and fit to non-linear regression models for computation of cellular IC₅₀ values.

### In Vitro Drug Synergy Assays for Mutant BCR-ABL1 Cells Lines

For Ba/F3 cells expressing BCR-ABL1 wild-type, T315I, or a subset of compound mutants, Compound A will be evaluated in similar cell proliferation assays to those described above but in combination with the approved ATP-site inhibitors imatinib, dasatinib, or ponatinib to determine potential for synergistic inhibition. Cells will be tested against each inhibitor alone and across a dose matrix of all possible dose combinations. Normalized MTS absorbance data will be further analyzed using the synergyfinder R package to quantify potential synergy by multiple models (e.g., highest single agent (HSA), Bliss, zero interaction potency (ZIP)). Similar dose matrices will be tested on Ba/F3 parental cells to confirm combined off-target toxicity.

### Cell-Based Accelerated Resistance Screens

Compound A will be profiled using in vitro cell-based mutagenesis screens. Briefly, Ba/F3 BCR-ABL1 cells will be treated overnight with ENU (to induce random mutations), resuspended in fresh culture media, and plated in the presence of graded concentrations of inhibitor. Wells with outgrowth are expanded, harvested, subjected to DNA isolation and PCR amplification of the BCR-ABL1 kinase domain (approximately ABL1a residues 235-530), and Sanger sequenced for mutations. A similar mutagenesis resistance screen will be performed starting from Ba/F3 cells expressing BCR-ABL1 T315I to identify potential compound mutant resistance mechanisms, and also performed in combination with ponatinib to determine the potential for suppression of compound mutant outgrowth.

### Primary CML Patient Cell Ex Vivo Assay

Primary mononuclear cells from 3-4 CML patients with either newly diagnosed or a T315I resistant mutation will be thawed and treated either overnight or for 3 days with concentrations of Compound A; imatinib treatment will be included as a control. Overnight-treated cells will be lysed and subjected to immunoblot analysis for pCRKL levels, and 3-day-treated cells will be analyzed with respect to effect on viability and/or annexin-V apoptotic induction.

### Example 4: Characterization of Compound a Against Native and Mutant BCR:ABL-1

A study will be performed to determine the activity of Compound A alone and in combination with dasatinib or ponatinib in vitro. BaF/3 cells expressing native BCR::ABL1, BCR::ABL1 single mutants mutants (F359V, F359C, F359I, H396R, E255V, T315I, A337V, A344P, P465S, V468F), and BCR::ABL1 compound mutants (G250E/T315I, Y253H/T315I, E255V/T315I, H396R/T315I, E255V/V299L, T315M, Y253H/F317L, A337V/T315I, A344P/T315I, P465S/T315I, V468F/T315I) will be cultured in the presence of graded concentrations (4-log range, starting from 0.1 nM) of Compound A and dasatinib or Compound A and ponatinib, respectively. Assays will be performed in triplicate, and viable cells quantified by MTS assay. Synergy will be analyzed using Combenefit and Synergy finder platforms, interactive platform allowing to collect all experimental replicates. Synergy scores will be determined using Bliss and ZIP reference models. Data will be reported in tables and graphs.

Determine the activity of Compound A alone and in combination with dasatinib or ponatinib on BCR::ABL1 signaling. pSTAT5/STAT5 will be quantified by immunoblot in BaF/3 cells expressing native or mutant BCR:ABL1 treated with inhibitors using a stable concentration of Compound A and a 4-log range for dasatinib and ponatinib, respectively. Data will be reported as immunoblotting images, FACS histograms and graphs.

Assess the in vivo activity of Compound A single agent or combined with dasatinib or ponatinib in a retroviral transduction/transplantation model of CML, using native, and E255V/T315I mutant BCR::ABL1. Bone marrow from 5-fluorouracil treated Balb/c mice will be transduced with BCR::ABL1-GFP retrovirus and injected cells in lethally irradiated syngeneic recipients. Upon demonstration of leukemic engraftment (GFP+ cells detected in the blood by FACS) mice (N=10/group) will be started on Compound A, dasatinib, ponatinib, Compound A plus dasatinib, Compound A plus ponatinib, or vehicle. Mice will be monitored by daily inspection and weighing, weekly complete blood counts and FACS+ for GFP+ blood cells. At development of distress or at 6 weeks (end of treatment, EOT), mice will be sacrificed and subjected to detailed autopsy, histology of involved organs, and quantification of leukemia burden by flow cytometry. Treatment arms will be compared for survival using Kaplan-Meyer statistics and for differences in continues variables by T-test.

1. A method of treating cancer in a subject in need thereof, comprising administering a BCR:ABL1 inhibitor in combination with a second tyrosine-kinase inhibitor (TKI), wherein the BCR:ABL1 inhibitor is a compound of formula (I): 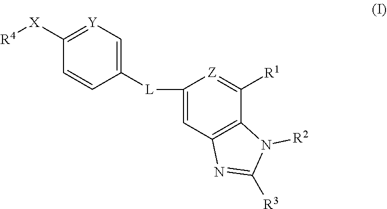
or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷,
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-.

2-3. (canceled)

4. A method of treating leukemia in a subject in need thereof comprising administering to the patient a therapeutically effective amount of a BCR:ABL-1 inhibitor of Formula (I): 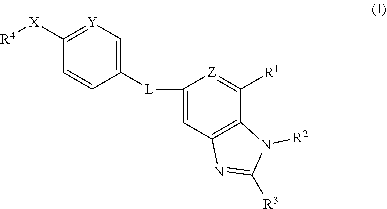
or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷;
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-;
in combination with a therapeutically effective amount of a second tyrosine-kinase inhibitor, wherein the leukemia is chronic myeloid leukemia (CML), acute myeloid leukemia (AML), or acute lymphoblastic leukemia (ALL).

5. (canceled)

6. The method of claim 1, wherein the method comprises administering the BCR:ABL1 inhibitor of Formula (I) and the second TKI to the subject at a ratio of about 1:1, about 1:2, about 1:3, about 1:4, about 1:5, about 2:1, about 2:3, about 2:5, about 3:1, about 3:2, about 3:4, about 3:5, about 4:1, about 4:3, or about 4:5.

7. The method of claim 1, wherein in the BCR:ABL1 inhibitor of Formula (I) is (R)—N-(4-(chlorodifluoromethoxy)phenyl)-2-(difluoromethyl)-1-(1-hydroxypropan-2-yl)-7-(pyrimidin-5-yl)-1H-benzo[d]imidazole-5-carboxamide (Compound A): 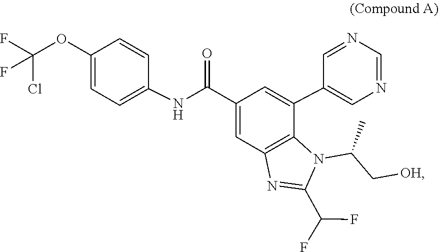 [SMILES: C[C@H](CO)n1c(C(F)F)nc2cc(C(=O)Nc3ccc(OC(F)(F)Cl)cc3)cc(-c3cncnc3)c21]
or a pharmaceutically acceptable salt thereof.

8. The method of claim 1, wherein the subject has previously been treated with asciminib.

9. The method of claim 1, wherein the subject has previously been treated with a combination of asciminib and a second TKI.

10. The method of claim 1, wherein the second TKI binds to the active site of the BCR:ABL-1.

11. The method of claim 1, wherein the second TKI is selected from imatinib, nilotinib, dasatinib, bosutinib, ponatinib and bafetinib.

12. The method of claim 1, wherein the second TKI is selected from ponatinib and dasatinib.

13. The method of claim 1, wherein the cancer is lymphoma or leukemia.

14. The method of claim 1, wherein the lymphoma or leukemia is chromic myeloid leukemia (CML) or chronic lymphocytic leukemia (CLL).

15. The method of claim 1, wherein the method further comprises the step of:
determining the DNA sequence of the BCR:ABL1 gene expressed by the subject; and/or
determining the amino acid sequence of the BCR:ABL1 protein expressed by the subject.

16. The method of claim 1, wherein the subject expresses wild-type BCR:ABL-1.

17. The method of claim 16, wherein the wild-type form of BCR:ABL-1 has the amino acid sequence of SEQ ID NO: 1.

18. The method of claim 1, wherein the subject expresses a mutant form of BCR:ABL-1.

19. The method of claim 1, wherein the mutant form of BCR:ABL-1 contains;
(i) a single amino acid mutation selected from F359V, F359C, F359I, H396R, E255V, T315I, A337V, A344P, P465S, T315M, and V468F; or
(ii) multiple amino acid mutations selected from (i) G250E and T315I, (ii) Y253H and T315I, (iii) E255V and T315I, (iv) H396R and T315I, (v) E255V and V299L, (vi) Y253H and F317L, (vii) A337V and T315I, (viii) A344P and T315I, (ix) P465S and T315I, and (x) V468F and T315I.

20. (canceled)

21. The method of claim 19, wherein the mutant form of BCR:ABL-1 has the amino acid sequence of any one of SEQ ID NOS 2-22.

22. A combination comprising a BCR:ABL1 inhibitor and a tyrosine-kinase inhibitor, wherein the BCR:ABL1 inhibitor is a compound of formula (I): 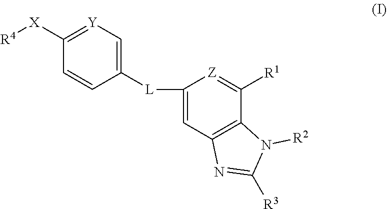
or a tautomer or an N-oxide thereof, or a pharmaceutically acceptable salt of any of the foregoing, wherein:
L is —NH—CO—, —CO—NH—, —NH—SO₂—, or —SO₂—NH—;
R¹ is optionally substituted C₆-C₁₀ aryl, optionally substituted 5-10 membered heteroaryl, optionally substituted 4-10 membered heterocycle, C(O)NR⁶R⁷, S(O)₂NR⁶R⁷, NR⁶COR⁷, NR⁶SO₂R⁷, or C(O)OR⁶;
R² is H, optionally substituted C₁-C₆ alkyl, optionally substituted C₃-C₈ cycloalkyl, optionally substituted 4-10 membered heterocycloalkyl, optionally substituted C₂-C₆ alkenyl, optionally substituted C₂-C₆ alkynyl, optionally substituted C₆-C₁₀ aryl, or optionally substituted 5-10 membered heteroaryl;
R³ is H, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, optionally substituted heteroaryl, OR⁶, or NR⁶R⁷;
or R² and R³ together with the intervening atoms form optionally substituted C₃-C₈ cycloalkyl or optionally substituted 4-10 membered heterocycloalkyl;
R⁴ is optionally substituted C₁-C₆ alkyl, optionally substituted C₂-C₆ alkenyl, or optionally substituted C₂-C₆ alkynyl;
X is O or S;
Y is CH, C—(C₁-C₂ alkyl), or C-halo or N;
Z is CR⁵ or N;
R⁵ is H or halogen;
R⁶ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl; and
R⁷ is H, optionally substituted C₁-C₆ alkyl, optionally substituted C₃-C₈ cycloalkyl, optionally substituted 4-10 membered heterocycloalkyl, optionally substituted C₆-C₁₀ aryl, or optionally substituted 5-10 membered heteroaryl;
or R⁶ and R⁷ together with the nitrogen to which they are attached form an optionally substituted 4-7 membered heterocycle,
provided that the compound is other than (i) 1H-Benzimidazole-7-carboxylic acid, 5-[[(4-methoxyphenyl)sulfonyl]amino]-1-methyl- or (ii) 1H-Benzimidazole-7-carboxylic acid, 5-[[(4-ethoxyphenyl)sulfonyl]amino]-1-methyl-;
and at least one pharmaceutically acceptable excipient.

23-28. (canceled)

29. The method of claim 1, wherein the subject previously received treatment with at least two tyrosine-kinase inhibitors.

30. The method of claim 1, wherein the subject previously received treatment with at least one allosteric tyrosine-kinase inhibitor.

31. The method of claim 1, wherein the subject relapsed on previous treatment.

32. The method of claim 1, wherein the subject was refractory to previous treatment.

33. The method of claim 1, wherein the subject relapsed and was refractory to previous treatment.


SEQUENCE LISTING (22 sequences)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 2 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNVIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 3 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNCIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 4 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNIIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 5 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTARAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 6 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGVVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 7 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 8 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNVVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 9 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMPTQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 10 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCSEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 11 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIMEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 12 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKFYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 13 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGEGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 14 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQHGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 15 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGVVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 16 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTARAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 17 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGVVYEGVWKKYSLTVAVKTLKEDTMEVEEFLKEAAVMKEIKHPNLLQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 18 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQHGEVYEGVWKKYSLTVAVKTLKEDTMEVEEFLKEAAVMKEIKHPNLVQLLGVCTREPPFYIITELMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 19 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNVVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 20 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMPTQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 21 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCSEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 22 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKFYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR